(19) United States
(12) Patent Application Publication
(10) Pub. No.: US 2022/0346357 A1
(43) Pub. Date: Nov. 3, 2022

(54) GENETICALLY MODIFIED NON-HUMAN ANIMAL WITH HUMAN OR CHIMERIC TNFR2

(71) Applicant: Biocytogen Pharmaceuticals (Beijing) Co., Ltd., Beijing (CN)

(72) Inventors: Yuelei Shen, Beijing (CN); Rui Huang, Beijing (CN); Yanan Guo, Beijing (CN); Yang Bai, Beijing (CN); Lei Zhao, Beijing (CN); Jiawei Yao, Beijing (CN); Chaoshe Guo, Beijing (CN); Meiling Zhang, Beijing (CN)

(21) Appl. No.: 17/640,592

(22) PCT Filed: Sep. 4, 2020
(86) PCT No.: PCT/CN2020/113618

§ 371 Date: Mar. 4, 2022

(30) Foreign Application Priority Data
Sep. 6, 2019 (CN) 201910842697.X
Jan. 16, 2020 (CN) 202010049072.0

(51) Int. Cl. A01K 67/027; A61K 49/00; A61P 35/00; C07K 14/525; C07K 14/705; C07K 14/715; C12N 15/85

(52) U.S. Cl. CPC A01K 67/0278; A61K 49/0008; A61P 35/00; C07K 14/525; C07K 14/70521; C07K 14/70575; C07K 14/70578; C07K 14/7151; C12N 15/8509; A01K 2207/15; A01K 2217/072; A01K 2227/105; A01K 2267/03; C07K 2319/00

(57) ABSTRACT

The present disclosure relates to genetically modified non-human animals that express a human or chimeric (e.g., humanized) TNFR2, and methods of use thereof.

### CLAIM OF PRIORITY

This application claims the benefit of Chinese Patent Application App. No. 201910842697.X, filed on Sep. 6, 2019, and Chinese Patent Application App. No. 202010049072.0, filed on Jan. 16, 2020. The entire contents of the foregoing are incorporated herein by reference.

### TECHNICAL FIELD

This disclosure relates to genetically modified animal expressing human or chimeric (e.g., humanized) TNFR2, and methods of use thereof.

### BACKGROUND

The immune system has developed multiple mechanisms to prevent deleterious activation of immune cells. One such mechanism is the intricate balance between positive and negative costimulatory signals delivered to immune cells. Targeting the stimulatory or inhibitory pathways for the immune system is considered to be a potential approach for the treatment of various diseases, e.g., cancers and autoimmune diseases.

The traditional drug research and development for these stimulatory or inhibitory receptors typically use in vitro screening approaches. However, these screening approaches cannot provide the body environment (such as tumor microenvironment, stromal cells, extracellular matrix components and immune cell interaction, etc.), resulting in a higher rate of failure in drug development. In addition, in view of the differences between humans and animals, the test results obtained from the use of conventional experimental animals for in vivo pharmacological test may not reflect the real disease state and the interaction at the targeting sites, resulting in that the results in many clinical trials are significantly different from the animal experimental results. Therefore, the development of humanized animal models that are suitable for human antibody screening and evaluation will significantly improve the efficiency of new drug development and reduce the cost for drug research and development.

### SUMMARY

This disclosure is related to an animal model with human TNFR2 or chimeric TNFR2. The animal model can express human TNFR2 or chimeric TNFR2 (e.g., humanized TNFR2) protein in its body. It can be used in the studies on the function of TNFR2 gene, and can be used in the screening and evaluation of anti-human TNFR2 antibodies. In addition, the animal models prepared by the methods described herein can be used in drug screening, pharmacodynamics studies, treatments for immune-related diseases (e.g., autoimmune disease), and cancer therapy for human TNFR2 target sites; they can also be used to facilitate the development and design of new drugs, and save time and cost. In summary, this disclosure provides a powerful tool for studying the function of TNFR2 protein and a platform for screening cancer drugs.

In one aspect, the disclosure is related to a genetically-modified, non-human animal whose genome comprises at least one chromosome comprising a sequence encoding a human or chimeric tumor necrosis factor receptor 2 (TNFR2).

In some embodiments, the sequence encoding the human or chimeric TNFR2 is operably linked to an endogenous regulatory element at the endogenous TNFR2 gene locus in the at least one chromosome.

In some embodiments, the sequence encoding a human or chimeric TNFR2 comprises a sequence encoding an amino acid sequence that is at least 70%, 75%, 80%, 85%, 90%, 95%, 99%, or 100% identical to human TNFR2 (NP 001057.1 (SEQ ID NO: 4)).

In some embodiments, the sequence encoding a human or chimeric TNFR2 comprises a sequence encoding an amino acid sequence that is at least 70%, 75%, 80%, 85%, 90%, 95%, 99%, or 100% identical to SEQ ID NO: 9.

In some embodiments, the sequence encoding a human or chimeric TNFR2 comprises a sequence that is at least 70%, 75%, 80%, 85%, 90%, 95%, 99%, or 100% identical to amino acids 33-259 of SEQ ID NO: 4.

In some embodiments, the animal is a mammal, e.g., a monkey, a rodent, or a mouse. In some embodiments, the animal is a mouse.

In some embodiments, the animal does not express endogenous TNFR2.

In some embodiments, the animal has one or more cells expressing human or chimeric TNFR2.

In some embodiments, the animal has one or more cells expressing human or chimeric TNFR2, and a human TNFα can bind to the expressed human or chimeric TNFR2. In some embodiments, the animal has one or more cells expressing human or chimeric TNFR2, and an endogenous TNFα can bind to the expressed human or chimeric TNFR2.

In one aspect, the disclosure is related to a genetically-modified, non-human animal. In some embodiments, the genome of the animal comprises a replacement of a sequence encoding a region of endogenous TNFR2 with a sequence encoding a corresponding region of human TNFR2 at an endogenous TNFR2 gene locus.

In some embodiments, the sequence encoding the corresponding region of human TNFR2 is operably linked to an endogenous regulatory element at the endogenous TNFR2 locus, and one or more cells of the animal expresses a chimeric TNFR2.

In some embodiments, the animal does not express endogenous TNFR2.

In some embodiments, the replaced locus is the extracellular region of TNFR2.

In some embodiments, the animal has one or more cells expressing a chimeric TNFR2 having an extracellular region, a transmembrane region, and a cytoplasmic region. In some embodiments, the extracellular region comprises a sequence that is at least 50%, 60%, 70%, 80%, 90%, 95%, or 99% identical to the extracellular region of human TNFR2.

In some embodiments, the extracellular region of the chimeric TNFR2 has a sequence that has at least 10, 20, 30, 40, 50, 60, 70, 80, 90, or 100 contiguous amino acids that are identical to a contiguous sequence present in the extracellular region of human TNFR2.

In some embodiments, the animal is a mouse, and the sequence encoding the region of endogenous TNFR2 is exon 2, exon 3, exon 4, exon 5, and/or exon 6 of the endogenous mouse TNFR2 gene.

In some embodiments, the animal is heterozygous with respect to the replacement at the endogenous TNFR2 gene locus.

In some embodiments, the animal is homozygous with respect to the replacement at the endogenous TNFR2 gene locus.

In one aspect, the disclosure is related to a method for making a genetically-modified, non-human animal, comprising: replacing in at least one cell of the animal, at an endogenous TNFR2 gene locus, a sequence encoding a region of an endogenous TNFR2 with a sequence encoding a corresponding region of human TNFR2.

In some embodiments, the sequence encoding the corresponding region of human TNFR2 comprises exon 1, exon 2, exon 3, exon 4, exon 5, exon 6, exon 7, exon 8, exon 9, and/or exon 10 of a human TNFR2 gene.

In some embodiments, the sequence encoding the corresponding region of TNFR2 comprises exon 2, exon 3, exon 4, exon 5, and/or exon 6, or part thereof, of a human TNFR2 gene.

In some embodiments, the sequence encoding the corresponding region of human TNFR2 encodes amino acids 33-259 of SEQ ID NO: 4.

In some embodiments, the region is located within the extracellular region of TNFR2.

In some embodiments, the animal is a mouse, and the endogenous TNFR2 locus is exon 2, exon 3, exon 4, exon 5, exon 6, exon 7, exon 8, exon 9, and/or exon 10 of the mouse TNFR2 gene.

In some embodiments, the animal is a mouse, and the endogenous TNFR2 locus is exon 2, exon 3, exon 4, exon 5, and/or exon 6 of the mouse TNFR2 gene.

In one aspect, the disclosure is related to a non-human animal comprising at least one cell comprising a nucleotide sequence encoding a chimeric TNFR2 polypeptide. In some embodiments, the chimeric TNFR2 polypeptide comprises at least 50 contiguous amino acid residues that are identical to the corresponding contiguous amino acid sequence of a human TNFR2. In some embodiments, the animal expresses the chimeric TNFR2.

In some embodiments, the chimeric TNFR2 polypeptide has at least 50 contiguous amino acid residues that are identical to the corresponding contiguous amino acid sequence of a human TNFR2 extracellular region.

In some embodiments, the chimeric TNFR2 polypeptide comprises a sequence that is at least 90%, 95%, or 99% identical to amino acids 33-259 of SEQ ID NO: 4.

In some embodiments, the nucleotide sequence is operably linked to an endogenous TNFR2 regulatory element of the animal.

In some embodiments, the chimeric TNFR2 polypeptide comprises an endogenous TNFR2 transmembrane region and/or an endogenous TNFR2 cytoplasmic region.

In some embodiments, the nucleotide sequence is integrated to an endogenous TNFR2 gene locus of the animal.

In some embodiments, the chimeric TNFR2 has at least one mouse TNFR2 activity and/or at least one human TNFR2 activity.

In one aspect, the disclosure is related to a method of making a genetically-modified mouse cell that expresses a chimeric TNFR2, the method comprising: replacing at an endogenous mouse TNFR2 gene locus, a nucleotide sequence encoding a region of mouse TNFR2 with a nucleotide sequence encoding a corresponding region of human TNFR2, thereby generating a genetically-modified mouse cell that includes a nucleotide sequence that encodes the chimeric TNFR2. In some embodiments, the mouse cell expresses the chimeric TNFR2.

In some embodiments, the chimeric TNFR2 comprises: an extracellular region of human TNFR2 comprising a human or mouse signal peptide sequence; and a transmembrane and/or a cytoplasmic region of mouse TNFR2.

In some embodiments, the nucleotide sequence encoding the chimeric TNFR2 is operably linked to an endogenous TNFR2 regulatory region, e.g., promoter.

In some embodiments, the animal further comprises a sequence encoding an additional human or chimeric protein. In some embodiments, the additional human or chimeric protein is tumor necrosis factor alpha (TNFα), programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (OX40).

In some embodiments, the animal or mouse further comprises a sequence encoding an additional human or chimeric protein. In some embodiments, the additional human or chimeric protein is TNFα, PD-1, CTLA-4, LAG-3, BTLA, PD-L1, CD27, CD28, CD47, CD137, CD154, TIGIT, TIM-3, GITR, SIRPα or OX40.

In one aspect, the disclosure is related to a method of determining effectiveness of an anti-TNFR2 antibody for the treatment of cancer, comprising: a) administering the anti-TNFR2 antibody to the animal as described herein, and in some embodiments, the animal has a tumor; and b) determining the inhibitory effects of the anti-TNFR2 antibody to the tumor.

In some embodiments, the tumor comprises one or more cells that express TNFR2. In some embodiments, the TNFR2 is a human TNFR2, a humanized TNFR2, or an endogenous TNFR2.

In some embodiments, the tumor comprises one or more cancer cells that are injected into the animal.

In some embodiments, determining the inhibitory effects of the anti-TNFR2 antibody to the tumor involves measuring the tumor volume in the animal.

In some embodiments, the tumor cells are breast cancer cells, colon cancer cells, cervical cancer cells, fibrosarcoma, liver cancer cells, lung cancer cells, melanoma cells, ovarian cancer cells, renal cancer cells, skin cancer cells, plasmacytoma, lymphoma, or leukemia.

In one aspect, the disclosure is related to a method of determining effectiveness of an anti-TNFR2 antibody and an additional therapeutic agent for the treatment of a tumor, comprising: a) administering the anti-TNFR2 antibody and the additional therapeutic agent to the animal as described herein, and in some embodiments, the animal has a tumor; and b) determining the inhibitory effects on the tumor.

In some embodiments, the animal further comprises a sequence encoding a human or chimeric programmed cell death protein 1 (PD-1). In some embodiments, the animal further comprises a sequence encoding a human or chimeric programmed death-ligand 1 (PD-L1).

In some embodiments, the additional therapeutic agent is an anti-PD-1 antibody or an anti-PD-L1 antibody.

In some embodiments, the tumor comprises one or more tumor cells that express TNFR2, PD-1 or PD-L1.

In some embodiments, the tumor is caused by injection of one or more cancer cells into the animal.

In some embodiments, determining the inhibitory effects of the treatment involves measuring the tumor volume in the animal.

In some embodiments, the animal has breast cancer, colon cancer, cervical cancer, fibrosarcoma, liver cancer, lung cancer, non-small cell lung cancer (NSCLC), melanoma, ovarian cancer, renal cancer, skin cancer, plasmacytoma, lymphoma, and/or leukemia.

In one aspect, the disclosure is related to a method of determining effectiveness of an anti-TNFR2 antibody for treating an autoimmune disorder, comprising: a) administering the anti-TNFR2 antibody to the animal as described herein, and in some embodiments, the animal has the autoimmune disorder; and b) determining effects of the anti-TNFR2 antibody for treating the auto-immune disease.

In some embodiments, the autoimmune disorder is rheumatoid arthritis, Crohn's disease, systemic lupus erythematosus, ankylosing spondylitis, inflammatory bowel diseases (IBD), ulcerative colitis, and/or scleroderma.

In one aspect, the disclosure is related to a method of determining effectiveness of an anti-TNFR2 antibody for treating an immune disorder, comprising: a) administering the anti-TNFR2 antibody to the animal as described herein, and in some embodiments, the animal has the immune disorder; and b) determining effects of the anti-TNFR2 antibody for treating the immune disease.

In some embodiments, the immune disorder is allergy, asthma, and/or atopic dermatitis.

In one aspect, the disclosure is related to a protein comprising an amino acid sequence, and in some embodiments, the amino acid sequence is one of the following:
(a) an amino acid sequence set forth in SEQ ID NO: 9;
(b) an amino acid sequence that is at least 90% identical to SEQ ID NO: 9;
(c) an amino acid sequence that is at least 91%, 92%, 93%, 94%, 95%, 96%, 97%, 98%, or 99% identical to SEQ ID NO: 9;
(d) an amino acid sequence that is different from the amino acid sequence set forth in SEQ ID NO: 9 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 9.

In one aspect, the disclosure is related to a nucleic acid comprising a nucleotide sequence, and in some embodiments, the nucleotide sequence is one of the following:
(a) a sequence that encodes the protein as described herein;
(b) SEQ ID NO: 7, SEQ ID NO: 8, SEQ ID NO: 10, or SEQ ID NO: 11;
(c) a sequence that is at least 90% identical to SEQ ID NO: 7, 8, 10, or 11;
(d) a sequence that is at least 91%, 92%, 93%, 94%, 95%, 96%, 97%, 98%, or 99% identical to SEQ ID NO: 7, 8, 10, or 11; and
(e) a sequence that is at least 91%, 92%, 93%, 94%, 95%, 96%, 97%, 98%, or 99% identical to SEQ ID NO: 7, 8, 10, or 11.

In one aspect, the disclosure is related to a cell comprising the protein and/or the nucleic acid as described herein.

In one aspect, the disclosure is related to an animal comprising the protein and/or the nucleic acid as described herein.

In another aspect, the disclosure also provides a genetically-modified, non-human animal whose genome comprise a disruption in the animal's endogenous TNFR2 gene, wherein the disruption of the endogenous TNFR2 gene comprises deletion of exon 1, exon 2, exon 3, exon 4, exon 5, exon 6, exon 7, exon 8, exon 9, and/or exon 10, or part thereof of the endogenous TNFR2 gene.

In some embodiments, the disruption of the endogenous TNFR2 gene comprises deletion of one or more exons or part of exons selected from the group consisting of exon 2, exon 3, exon 4, exon 5, and exon 6 of the endogenous TNFR2 gene.

In some embodiments, the disruption of the endogenous TNFR2 gene further comprises deletion of one or more introns or part of introns selected from the group consisting of intron 2, intron 3, intron 4, and intron 5 of the endogenous TNFR2 gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, 300, 350, 400, 450, 500, 550, 600, 650, or more nucleotides.

In some embodiments, the disruption of the endogenous TNFR2 gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, or 300 nucleotides of exon 1, exon 2, exon 3, exon 4, exon 5, exon 6, exon 7, exon 8, exon 9, and/or exon 10 (e.g., deletion of at least 300 nucleotides starting from exon 2 to exon 6).

The disclosure further relates to a TNFR2 genomic DNA sequence of a humanized mouse, a DNA sequence obtained by a reverse transcription of the mRNA obtained by transcription thereof is consistent with or complementary to the DNA sequence; a construct expressing the amino acid sequence thereof; a cell comprising the construct thereof; a tissue comprising the cell thereof.

The disclosure further relates to the use of the non-human mammal or an offspring thereof, or the tumor bearing non-human mammal, the animal model generated through the method as described herein in the development of a product related to an immunization processes of human cells, the manufacture of a human antibody, or the model system for a research in pharmacology, immunology, microbiology and medicine.

The disclosure also relates to the use of the non-human mammal or an offspring thereof, or the tumor bearing non-human mammal, the animal model generated through the method as described herein in the production and utilization of an animal experimental disease model of an immunization processes involving human cells, the study on a pathogen, or the development of a new diagnostic strategy and/or a therapeutic strategy.

The disclosure further relates to the use of the non-human mammal or an offspring thereof, or the tumor bearing non-human mammal, the animal model generated through the methods as described herein, in the screening, verifying, evaluating or studying the TNFR2 gene function, human TNFR2 antibodies, the drugs or efficacies for human TNFR2 targeting sites, and the drugs for immune-related diseases and antitumor drugs.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a schematic diagram showing mouse TNFR2 gene locus.

FIG. 1B is a schematic diagram showing human TNFR2 gene locus.

FIG. 2 is a schematic diagram showing humanized TNFR2 gene locus.

FIG. 3 is a schematic diagram showing an TNFR2 gene targeting strategy.

FIG. 4 shows Southern Blot results. WT is wild-type.

FIG. 5 is a schematic diagram showing the FRT recombination process. Positive heterozygous mice were mated with the Flp mice.

FIG. 6A shows PCR identification result of samples collected from tails of F1 generation mice. Primers WT-F/WT-R were used for amplification. WT is wild-type C57BL/6 mice. H₂O is a blank control. PC1 and PC2 are positive controls. M is marker. A mouse labelled F1-1 was identified as a positive clone.

FIG. 6B shows PCR identification result of samples collected from tails of F1 generation mice. Primers WT-F/Mut-R were used for amplification. WT is wild-type C57BL/6 mice. H₂O is a blank control. PC1 and PC2 are positive controls. M is marker. A mouse labelled F1-1 was identified as a positive clone.

FIG. 6C shows PCR identification result of samples collected from tails of F1 generation mice. Primers Frt-F/Frt-R were used for amplification. WT is wild-type C57BL/6 mice. H₂O is a blank control. PC is a positive control. M is marker. A mouse labelled F1-1 was identified as a positive clone.

FIG. 6D shows PCR identification result of samples collected from tails of F1 generation mice. Primers Flp-F2/Flp-R2 were used for amplification. WT is wild-type C57BL/6 mice. H₂O is a blank control. PC1 and PC2 are positive controls. M is marker. A mouse labelled F1-1 was identified as a positive clone.

FIG. 7A is a flow cytometry result of spleen cells from wild-type C57BL/6 mice. The cells were labeled with anti-mCD19 FITC, then with an isotype control antibody (ISO).

FIG. 7B is a flow cytometry result of spleen cells from TNFR2 gene humanized heterozygous mice (B-hTNFR2 (H/+) mice). The cells were labeled with anti-mCD19 FITC, then with an isotype control antibody (ISO).

FIG. 7C is a flow cytometry result of spleen cells from wild-type C57BL/6 mice. The cells were labeled with anti-mCD19 FITC, then with anti-mTNFR2 PE.

FIG. 7D is a flow cytometry result of spleen cells from TNFR2 gene humanized heterozygous mice (B-hTNFR2 (H/+) mice). The cells were labeled with anti-mCD19 FITC, then with anti-mTNFR2 PE.

FIG. 7E is a flow cytometry result of spleen cells from wild-type C57BL/6 mice. The cells were labeled with anti-mCD19 FITC, then with anti-hTNFR2 PE.

FIG. 7F is a flow cytometry result of spleen cells from TNFR2 gene humanized heterozygous mice (B-hTNFR2 (H/+) mice). The cells were labeled with anti-mCD19 FITC, then with anti-hTNFR2 PE.

FIG. 8A is a flow cytometry result of spleen cells from wild-type C57BL/6 mice. The cells were labeled with anti-mTcRβ PerCP/Cy5.5, then with an isotype control antibody (ISO).

FIG. 8B is a flow cytometry result of spleen cells from TNFR2 gene humanized heterozygous mice (B-hTNFR2 (H/+) mice). The cells were labeled with anti-mTcRβ PerCP/Cy5.5, then with an isotype control antibody (ISO).

FIG. 8C is a flow cytometry result of spleen cells from wild-type C57BL/6 mice. The cells were labeled with anti-mTcRβ PerCP/Cy5.5, then with anti-mTNFR2 PE.

FIG. 8D is a flow cytometry result of spleen cells from TNFR2 gene humanized heterozygous mice (B-hTNFR2 (H/+) mice). The cells were labeled with anti-mTcRβ PerCP/Cy5.5, then with anti-mTNFR2 PE.

FIG. 8E is a flow cytometry result of spleen cells from wild-type C57BL/6 mice. The cells were labeled with anti-mTcRβ PerCP/Cy5.5, then with anti-hTNFR2 PE.

FIG. 8F is a flow cytometry result of spleen cells from TNFR2 gene humanized heterozygous mice (B-hTNFR2 (H/+) mice). The cells were labeled with anti-mTcRβ PerCP/Cy5.5, then with anti-hTNFR2 PE.

FIG. 9A is a flow cytometry result of spleen T cells from wild-type (WT) C57BL/6 mice. The cells were labelled with anti-mTcRβ PerCP/Cy5.5 and then with anti-mTNFR2 PE.

FIG. 9B is a flow cytometry result of spleen T cells from TNFR2 gene humanized homozygous (H/H) mice. The cells were labelled with anti-mTcRβ PerCP/Cy5.5 and then with anti-mTNFR2 PE.

FIG. 9C is a flow cytometry result of spleen T cells from wild-type (WT) C57BL/6 mice. The cells were labelled with anti-mTcRβ PerCP/Cy5.5 and then with anti-hTNFR2 PE.

FIG. 9D is a flow cytometry result of spleen T cells from TNFR2 gene humanized homozygous (H/H) mice. The cells were labelled with anti-mTcRβ PerCP/Cy5.5 and then with anti-hTNFR2 PE.

FIG. 10A is a flow cytometry result of spleen T cells from anti-mCD3 antibody-stimulated wild-type (WT) C57BL/6 mice. The cells were labelled with anti-mTcRβ PerCP/Cy5.5 and then with anti-mTNFR2 PE.

FIG. 10B is a flow cytometry result of spleen T cells from anti-mCD3 antibody-stimulated TNFR2 gene humanized homozygous (H/H) mice. The cells were labelled with anti-mTcRβ PerCP/Cy5.5 and then with anti-mTNFR2 PE.

FIG. 10C is a flow cytometry result of spleen T cells from anti-mCD3 antibody-stimulated wild-type (WT) C57BL/6 mice. The cells were labelled with anti-mTcRβ PerCP/Cy5.5 and then with anti-hTNFR2 PE.

FIG. 10D is a flow cytometry result of spleen T cells from anti-mCD3 antibody-stimulated TNFR2 gene humanized homozygous (H/H) mice. The cells were labelled with anti-mTcRβ PerCP/Cy5.5 and then with anti-hTNFR2 PE.

FIG. 11A is a flow cytometry result of spleen CD4+ T cells from wild-type (WT) C57BL/6 mice. The cells were labelled with anti-mCD4-BV421 and then with anti-mTNFR2 PE.

FIG. 11B is a flow cytometry result of spleen CD4+ T cells from TNFR2 gene humanized homozygous (H/H) mice. The cells were labelled with anti-mCD4-BV421 and then with anti-mTNFR2 PE.

FIG. 11C is a flow cytometry result of spleen CD4+ T cells from wild-type (WT) C57BL/6 mice. The cells were labelled with anti-mCD4-BV421 and then with anti-hTNFR2 PE.

FIG. 11D is a flow cytometry result of spleen CD4+ T cells from TNFR2 gene humanized homozygous (H/H) mice. The cells were labelled with anti-mCD4-BV421 and then with anti-hTNFR2 PE.

FIG. 12A is a flow cytometry result of spleen CD4+ T cells from anti-mCD3 antibody-stimulated wild-type (WT) C57BL/6 mice. The cells were labelled with anti-mCD4-BV421 and then with anti-mTNFR2 PE.

FIG. 12B is a flow cytometry result of spleen CD4+ T cells from anti-mCD3 antibody-stimulated TNFR2 gene humanized homozygous (H/H) mice. The cells were labelled with anti-mCD4-BV421 and then with anti-mTNFR2 PE.

FIG. 12C is a flow cytometry result of spleen CD4+ T cells from anti-mCD3 antibody-stimulated wild-type (WT) C57BL/6 mice. The cells were labelled with anti-mCD4-BV421 and then with anti-hTNFR2 PE.

FIG. 12D is a flow cytometry result of spleen CD4+ T cells from anti-mCD3 antibody-stimulated TNFR2 gene humanized homozygous (H/H) mice. The cells were labelled with anti-mCD4-BV421 and then with anti-hTNFR2 PE.

FIG. 13A is a flow cytometry result of spleen Treg cells from wild-type (WT) C57BL/6 mice. The cells were labelled with anti-mFoxp3-APC and then with anti-mTNFR2 PE.

FIG. 13B is a flow cytometry result of spleen Treg cells from TNFR2 gene humanized homozygous (H/H) mice. The cells were labelled with anti-mFoxp3-APC and then with anti-mTNFR2 PE.

FIG. 13C is a flow cytometry result of spleen Tregcells from wild-type (WT) C57BL/6 mice. The cells were labelled with anti-mFoxp3-APC and then with anti-hTNFR2 PE.

FIG. 13D is a flow cytometry result of spleen Tregcells from TNFR2 gene humanized homozygous (H/H) mice. The cells were labelled with anti-mFoxp3-APC and then with anti-hTNFR2 PE.

FIG. 14A is a flow cytometry result of spleen Tregcells from anti-mCD3 antibody-stimulated wild-type (WT) C57BL/6 mice. The cells were labelled with anti-mFoxp3-APC and then with anti-mTNFR2 PE.

FIG. 14B is a flow cytometry result of spleen Tregcells from anti-mCD3 antibody-stimulated TNFR2 gene humanized homozygous (H/H) mice. The cells were labelled with anti-mFoxp3-APC and then with anti-mTNFR2 PE.

FIG. 14C is a flow cytometry result of spleen Tregcells from anti-mCD3 antibody-stimulated wild-type (WT) C57BL/6 mice. The cells were labelled with anti-mFoxp3-APC and then with anti-hTNFR2 PE.

FIG. 14D is a flow cytometry result of spleen Treg cells from anti-mCD3 antibody-stimulated TNFR2 gene humanized homozygous (H/H) mice. The cells were labelled with anti-mFoxp3-APC and then with anti-hTNFR2 PE.

FIG. 15A is a flow cytometry result of spleen B cells from wild-type (WT) C57BL/6 mice. The cells were labelled with anti-mCD19 FITC and then with anti-mTNFR2 PE.

FIG. 15B is a flow cytometry result of spleen B cells from TNFR2 gene humanized homozygous (H/H) mice. The cells were labelled with anti-mCD19 FITC and then with anti-mTNFR2 PE.

FIG. 15C is a flow cytometry result of spleen B cells from wild-type (WT) C57BL/6 mice. The cells were labelled with anti-mCD19 FITC and then with anti-hTNFR2 PE.

FIG. 15D is a flow cytometry result of spleen B cells from TNFR2 gene humanized homozygous (H/H) mice. The cells were labelled with anti-mCD19 FITC and then with anti-hTNFR2 PE.

FIG. 16A is a flow cytometry result of spleen B cells from anti-mCD3 antibody-stimulated wild-type (WT) C57BL/6 mice. The cells were labelled with anti-mCD19 FITC and then with anti-mTNFR2 PE.

FIG. 16B is a flow cytometry result of spleen B cells from anti-mCD3 antibody-stimulated TNFR2 gene humanized homozygous (H/H) mice. The cells were labelled with anti-mCD19 FITC and then with anti-mTNFR2 PE.

FIG. 16C is a flow cytometry result of spleen B cells from anti-mCD3 antibody-stimulated wild-type (WT) C57BL/6 mice. The cells were labelled with anti-mCD19 FITC and then with anti-hTNFR2 PE.

FIG. 16D is a flow cytometry result of spleen B cells from anti-mCD3 antibody-stimulated TNFR2 gene humanized homozygous (H/H) mice. The cells were labelled with anti-mCD19 FITC and then with anti-hTNFR2 PE.

FIG. 17 shows percentages of immune cells in CD45+ spleen cells from wild-type C57BL/6 (WT) mice or TNFR2 gene humanized homozygous (H/H) mice.

FIG. 18 shows percentages of T cells in TCRβ+ spleen cells from wild-type C57BL/6 (WT) mice or TNFR2 gene humanized homozygous (H/H) mice.

FIG. 19 shows percentages of immune cells in CD45+ lymph node cells from wild-type C57BL/6 (WT) mice or TNFR2 gene humanized homozygous (H/H) mice.

FIG. 20 shows percentages of T cells in TCRβ+ lymph node cells from wild-type C57BL/6 (WT) mice or TNFR2 gene humanized homozygous (H/H) mice.

FIG. 21 shows relative TNFR2 gene expression as determined by q-PCR in wild-type C57BL/6 (WT) mice or TNFR2 gene humanized homozygous (H/H) mice.

FIG. 22 is a set of flow cytometry results showing CD3+ spleen T cell binding by anti-human TNFR2 antibodies Ab1, Ab2, and Ab3. Anti-mouse PD-1 antibody (mPD-1 Ab) is a positive control. IgG1 is an isotype control. A detailed protocol can be found in Table 5.

FIG. 23 shows the average weight of humanized TNFR2 homozygous mice that were xenografted with mouse colon cancer cells (MC38), and then treated with an isotype control antibody (G1), mPD-1 Ab (G2), anti-human TNFR2 antibodies Ab1 (G3), Ab2 (G4), and Ab3 (G5) at 10 mg/kg.

FIG. 24 shows the percentage change of average weight of humanized TNFR2 homozygous mice that were xenografted with mouse colon cancer cells (MC38), and then treated with an isotype control antibody (G1), mPD-1 Ab (G2), anti-human TNFR2 antibodies Ab1 (G3), Ab2 (G4), and Ab3 (G5) at 10 mg/kg.

FIG. 25 shows the average tumor volume of humanized TNFR2 homozygous mice that were xenografted with mouse colon cancer cells (MC38), and then treated with an isotype control antibody (G1), mPD-1 Ab (G2), anti-human TNFR2 antibodies Ab1 (G3), Ab2 (G4), and Ab3 (G5) at 10 mg/kg.

FIG. 26 shows the alignment between mouse TNFR2 amino acid sequence (NP_035740.2; SEQ ID NO: 2) and human TNFR2 amino acid sequence (NP_001057.1; SEQ ID NO: 4).

FIG. 27 shows the alignment between rat TNFR2 amino acid sequence (NP_569110.1; SEQ ID NO: 32 and human TNFR2 amino acid sequence (NP_001057.1; SEQ ID NO: 4).

### DETAILED DESCRIPTION

This disclosure relates to transgenic non-human animal with human or chimeric (e.g., humanized) TNFR2, and methods of use thereof.

Tumor necrosis factor (TNF) is widely accepted as a tumor-suppressive cytokine via its ubiquitous receptor TNF receptor 1 (TNFR1). The other receptor, TNFR2, is not only expressed on some tumor cells but also on suppressive immune cells, including regulatory T cells and myeloid-derived suppressor cells. In contrast to TNFR1, TNFR2 diverts the tumor-inhibiting TNF into a tumor-advocating factor. TNFR2 directly promotes the proliferation of some kinds of tumor cells. Also activating immunosuppressive cells, it supports immune escape and tumor development. Thus, TNFR2 antibodies can be potentially used as cancer therapies.

Experimental animal models are an indispensable research tool for studying the effects of these antibodies (e.g., TNFR2 antibodies). Common experimental animals include mice, rats, guinea pigs, hamsters, rabbits, dogs, monkeys, pigs, fish and so on. However, there are many differences between human and animal genes and protein sequences, and many human proteins cannot bind to the animal's homologous proteins to produce biological activity, leading to that the results of many clinical trials do not match the results obtained from animal experiments. A large number of clinical studies are in urgent need of better animal models. With the continuous development and maturation of genetic engineering technologies, the use of human cells or genes to replace or substitute an animal's endogenous similar cells or genes to establish a biological system or disease model closer to human, and establish the humanized experimental animal models (humanized animal model) has provided an important tool for new clinical approaches or means. In this context, the genetically engineered animal model, that is, the use of genetic manipulation techniques, the use of human normal or mutant genes to replace animal homologous genes, can be used to establish the genetically modified animal models that are closer to human gene systems. The humanized animal models have various important applications. For example, due to the presence of human or humanized genes, the animals can express or express in part of the proteins with human functions, so as to greatly reduce the differences in clinical trials between humans and animals, and provide the possibility of drug screening at animal levels.

Unless otherwise specified, the practice of the methods described herein can take advantage of the techniques of cell biology, cell culture, molecular biology, transgenic biology, microbiology, recombinant DNA and immunology. These techniques are explained in detail in the following literature, for examples: Molecular Cloning A Laboratory Manual, 2nd Ed., ed. By Sambrook, Fritsch and Maniatis (Cold Spring Harbor Laboratory Press: 1989); DNA Cloning, Volumes I and II (D. N. Glovered., 1985); Oligonucleotide Synthesis (M. J. Gaited., 1984); Mullis et al U. S. Pat. No. 4,683,195; Nucleic Acid Hybridization (B. D. Hames& S. J. Higginseds. 1984); Transcription And Translation (B. D. Hames& S. J. Higginseds. 1984); Culture Of Animal Cell (R. I. Freshney, Alan R. Liss, Inc., 1987); Immobilized Cells And Enzymes (IRL Press, 1986); B. Perbal, A Practical Guide To Molecular Cloning (1984), the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Caloseds., 1987, Cold Spring Harbor Laboratory); Immunochemical Methods In Cell And Molecular Biology (Mayer and Walker, eds., Academic Press, London, 1987); Hand book Of Experimental Immunology, Volumes V (D. M. Weir and C. C. Blackwell, eds., 1986); and Manipulating the Mouse Embryo, (Cold Spring Harbor Laboratory Press, Cold Spring Harbor, N.Y., 1986); each of which is incorporated herein by reference in its entirety.

### TNFR2

TNFR2 is a 75 kDa TNF superfamily receptor that transduces extracellular signals via receptor-associated cytoplasmic proteins. The signaling circuitry of TNFR2 is different from that of TNFR1; TNFR2 is not linked to a death domain but instead promotes NF-κB activation and cell growth. Most TNF superfamily receptors are expressed on all lymphoid and commonly on parenchymal cells. However, TNFR2 has limited expression in the immune system, is induced by the ligands TNF and interleukin-2 (IL-2), and is restricted to minor subpopulations of the lymphoid system, including potent Tregs, myeloid suppressor cells, endothelial cells, thymic T lymphocytes, microglia, oligodendrocytes, and select neurons during growth in mammals. This restricted expression of TNFR2 makes it an ideal drug target because systemic toxicity from an antibody-based therapy is less likely to occur. TNFR2 is an attractive candidate for additional reasons. In various human and murine cancers, abundant TNFR2-positive Tregs are found within the tumor microenvironment. Gene duplication and activating mutations in TNFR2 have been found in cancer. In addition, TNFR2 is present at a 10-fold higher density than TNFR1 in naturally occurring Tregs in human blood.

Both TNF receptor 1 (p55 or CD120a) and TNFR2 (p75 or CD120b) are type I transmembrane receptors. TNFR1 and TNFR2 have similar extracellular TNF-binding structures characterized by four repeated cysteine-rich domains (CRDs) (CRD1 also called pre-ligand binding assembly domain, CRD2, CRD3, and CRD4) but have different intracellular domains. Most critical for the diverse biological effects of the two receptor subtypes is the lack of the intracellular death domain in TNFR2. Hence, TNF promotes apoptosis via binding to TNFR1 but exerts pro-survival effects via TNFR2. After being engaged by extracellular TNF, TNFR1 recruits and clusters the adaptor protein TNFR1-associated death domain protein (TRADD) and the downstream caspases. This finally leads to programmed cell death. In contrast, activated TNFR2 results in recruitment of the TNF receptor-associated factor (TRAF) 2 and stimulates the pro-survival nuclear factor (NF)-κB pathway. TNFR2 has a high affinity to membrane-bound TNF and can deliver TNF to TNFR1.

Nuclear factor-κB is activated by both TNF receptor subtypes. Upon stimulation by its ligands including TNFα or lymphotoxin, TNFR1 forms a complex with the adaptor TRADD at the plasma membrane. TRAF2 is transported and clustered into the complex that recruits the cellular inhibitor of apoptosis 1 and 2 (cIAP1/2) proteins. Together with TRAF2, cIAP1/2 proteins degrade the TRADD-bound ubiquitinated receptor interacting protein (RIP) 1. Multiple ubiquitination of RIP1 and the NF-κB essential modulator (NEMO; also called IκB kinase (IKK)γ) engages the kinase TAK1 to the NEMO-containing IKK complex. IKKβ in the IKK complex becomes phosphorylated and phosphorylates the NF-κB inhibitor IκBα that is subsequently cleaved. Released NF-κB translocates into the nucleus and induces target gene expression.

A detailed description of TNFR2 and its function can be found, e.g., in Rothe et al., “The TNFR2-TRAF signaling complex contains two novel proteins related to baculoviral inhibitor of apoptosis proteins.” Cell 83.7 (1995): 1243-1252; Sheng et al., “TNF receptor 2 makes tumor necrosis factor a friend of tumors.” Frontiers in Immunology 9 (2018): 1170; Torrey et al., “Targeting TNFR2 with antagonistic antibodies inhibits proliferation of ovarian cancer cells and tumor-associated Tregs.” Science Signaling 10.462 (2017); Yang et al., “Role of TNF-TNF receptor 2 signal in regulatory T cells and its therapeutic implications.” Frontiers in Immunology 9 (2018): 784; each of which is incorporated by reference in its entirety.

In human genomes, TNFR2 gene (Gene ID: 7133) locus has ten exons, exon 1, exon 2, exon 3, exon 4, exon 5, exon 6, exon 7, exon 8, exon 9, and exon 10 (FIG. 1B). The TNFR2 protein also has an extracellular region, a transmembrane region, and a cytoplasmic region, and the signal peptide is located at the extracellular region of TNFR2. The nucleotide sequence for human TNFR2 mRNA is NM_001066.2 (SEQ ID NO: 3), and the amino acid sequence for human TNFR2 is NP_001057.1 (SEQ ID NO: 4). The location for each exon and each region in human TNFR2 nucleotide sequence and amino acid sequence is listed below:

TABLE 1

| | NM_001066.2 | NP_001057.1 |
|---|---|---|
| Human TNFR2 | 3682bp | 461aa |
| (approximate location) | SEQ ID NO: 3 | SEQ ID NO: 4 |
| Exon 1 | 1-167 | 1-26 |
| Exon 2 | 168 . . . 267 | 27-59 |
| Exon 3 | 268 . . . 396 | 60-102 |
| Exon 4 | 397-546 | 103-152 |
| Exon 5 | 547-640 | 153-184 |
| Exon 6 | 641-876 | 185-262 |
| Exon 7 | 877-954 | 263-288 |
| Exon 8 | 955-989 | 289-300 |
| Exon 9 | 990-1194 | 301-368 |
| Exon 10 | 1195-3675 | 369-461 |
| Signal peptide | 90-155 | 1-22 |
| Extracellular | 156-860 | 23-257 |
| Transmembrane region | 861-950 | 258-287 |
| Cytoplasmic | 951-1472 | 288-461 |
| Donor region in Example | 186-866 | 33-259 |

In mice, TNFR2 gene locus has ten exons, exon 1, exon 2, exon 3, exon 4, exon 5, exon 6, exon 7, exon 8, exon 9, and exon 10 (FIG. 1A). The mouse TNFR2 protein also has an extracellular region, a transmembrane region, and a cytoplasmic region, and the signal peptide is located at the extracellular region of TNFR2. The nucleotide sequence for mouse TNFR2 mRNA is NM_011610.3 (SEQ ID NO: 1), the amino acid sequence for mouse TNFR2 is NP_035740.2 (SEQ ID NO: 2). The location for each exon and each region in the mouse TNFR2 nucleotide sequence and amino acid sequence is listed below:

TABLE 2

| | NM_011610.3 | NP_035740.2 |
|---|---|---|
| Mouse TNFR2 | 4913 bp | 474aa |
| (approximate location) | SEQ ID NO: 1 | SEQ ID NO: 2 |
| Exon 1 | 1-147 | 1-26 |
| Exon 2 | 148-250 | 27-60 |
| Exon 3 | 251-379 | 61-103 |
| Exon 4 | 380-532 | 104-154 |
| Exon 5 | 533-626 | 155-186 |
| Exon 6 | 627-859 | 187-263 |
| Exon 7 | 860-937 | 264-289 |
| Exon 8 | 938-972 | 290-301 |
| Exon 9 | 973-1180 | 302-370 |
| Exon 10 | 1181-4913 | 371-474 |
| Signal peptide | 70-135 | 1-22 |
| Extracellular | 136-843 | 23-258 |
| Transmembrane region | 844-933 | 259-288 |
| Cytoplasmic | 934-1491 | 289-474 |
| Replaced region in Example | 166-849 | 33-260 |

The mouse TNFR2 gene (Gene ID: 21938) is located in Chromosome 4 of the mouse genome, which is located from 145212368 to 145246870 of NC_000070.6 (GRCm38.p4 (GCF_000001635.24)). The 5′-UTR is from 145,246,870 to 145,246,802, exon 1 is from 145,246,870 to 145,246,724, the first intron is from 145,246,7243 to 145,229,107, exon 2 is from 145,229,106 to 145,229,004, the second intron is from 145,229,003 to 145,227,597, exon 3 is from 145,227,596 to 145,227,468, the third intron is from 145,227,467 to 145,225,483, exon 4 is from 145,225,482 to 145,225,330, the fourth intron is from 145,225,329 to 145,224,906, exon 5 is from 145,224,905 to 145,224,812, the fifth intron is from 145,224,811 to 145,224,485, the exon 6 is from 145,224,484 to 145,224,252, the sixth intron is from 145,224,251 to 145,223,581, exon 7 is from 145,223,580 to 145,223,503, the seventh intron is from 145,223,502 to 145,222,989, exon 8 is from 145,222,988 to 145,222,954, the eighth intron is from 145,222,953 to 145,219,937, exon 9 is from 145,219,936 to 145,219,729, the ninth intron is from 145,219,728 to 145,216,101, exon 10 is from 145,216,100 to 145,213,463, the 3′-UTR is from 145,215,786 to 145,213,463, based on transcript NM_011610.3. All relevant information for mouse TNFR2 locus can be found in the NCBI website with Gene ID 21938, which is incorporated by reference herein in its entirety.

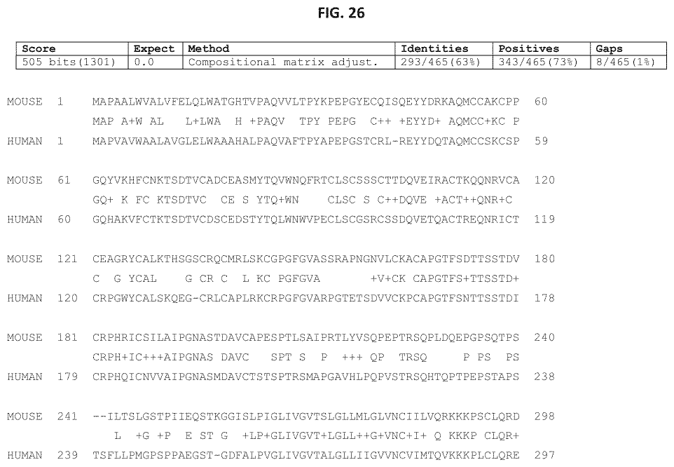

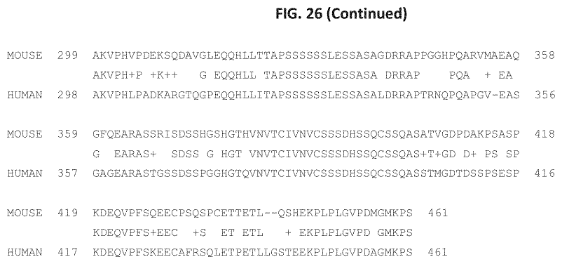

FIG. 26 shows the alignment between mouse TNFR2 amino acid sequence (NP_035740.2; SEQ ID NO: 2) and human TNFR2 amino acid sequence (NP_001057.1; SEQ ID NO: 4). Thus, the corresponding amino acid residue or region between human and mouse TNFR2 can be found in FIG. 26.

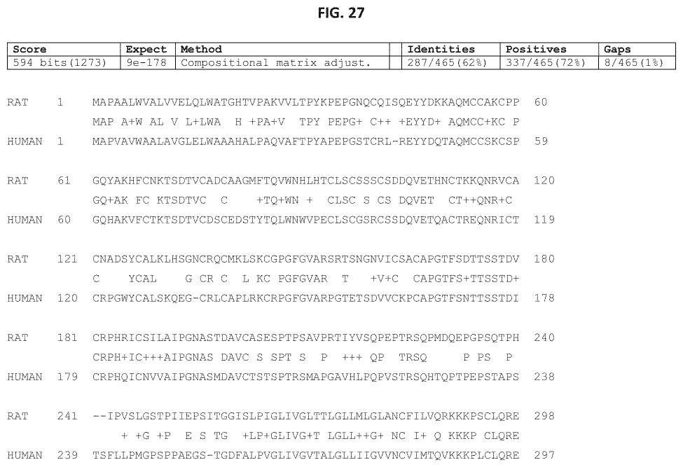

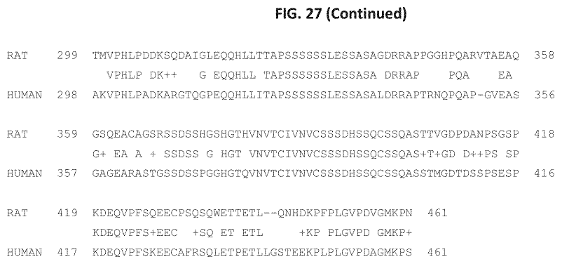

TNFR2 genes, proteins, and locus of the other species are also known in the art. For example, the gene ID for TNFR2 in Rattus norvegicus (rat) is 156767, the gene ID for TNFR2 in Macaca mulatta (Rhesus monkey) is 715454, the gene ID for TNFR2 in Equus caballus (horse) is 100055840, and the gene ID for TNFR2 in Sus scrofa (pig) is 100037306. The relevant information for these genes (e.g., intron sequences, exon sequences, amino acid residues of these proteins) can be found, e.g., in NCBI database, which is incorporated by reference herein in its entirety. FIG. 27 shows the alignment between rodent TNFR2 amino acid sequence (NP_569110.1; SEQ ID NO: 32) and human TNFR2 amino acid sequence (NP_001057.1; SEQ ID NO: 4). Thus, the corresponding amino acid residue or region between human and rodent TNFR2 can be found in FIG. 27.

The present disclosure provides human or chimeric (e.g., humanized) TNFR2 nucleotide sequence and/or amino acid sequences. In some embodiments, the entire sequence of mouse exon 1, exon 2, exon 3, exon 4, exon 5, exon 6, exon 7, exon 8, exon 9, exon 10, signal peptide, extracellular region, transmembrane region, and/or cytoplasmic region are replaced by the corresponding human sequence. In some embodiments, a “region” or “portion” of mouse exon 1, exon 2, exon 3, exon 4, exon 5, exon 6, exon 7, exon 8, exon 9, exon 10, signal peptide, extracellular region, transmembrane region, and/or cytoplasmic region are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, or 600 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, or 200 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, exon 9, exon 10, signal peptide, extracellular region, transmembrane region, or cytoplasmic region. In some embodiments, a region, a portion, or the entire sequence of mouse exon 1, exon 2, exon 3, exon 4, exon 5, exon 6, exon 7, exon 8, exon 9, and/or exon 10 (e.g., exon 2, exon 3, exon 4, exon 5, and exon 6) are replaced by a region, a portion, or the entire sequence of the human exon 1, exon 2, exon 3, exon 4, exon 5, exon 6, exon 7, exon 8, exon 9, and/or exon 10 (e.g., exon 2, exon 3, exon 4, exon 5, and exon 6) sequence.

In some embodiments, the present disclosure is related to a genetically-modified, non-human animal whose genome comprises a chimeric (e.g., humanized) TNFR2 nucleotide sequence. In some embodiments, the chimeric (e.g., humanized) TNFR2 nucleotide sequence encodes a TNFR2 protein comprising an extracellular region, a transmembrane region, a cytoplasmic region, and a signal peptide. In some embodiments, the extracellular region comprises the entire or part of human TNFR2 extracellular region. For example, the extracellular region described herein comprises a continuous 5-235 bp amino acid sequence that is identical to human TNFR2 extracellular region. In some embodiments, the signal peptide described herein is at least 80%, 85%, 90%, 95%, or 100% identical to amino acids 1-22 of SEQ ID NO: 2. In some embodiments, the signal peptide described herein is at least 80%, 85%, 90%, 95%, or 100% identical to amino acids 1-22 of SEQ ID NO: 4. In some embodiments, the cytoplasmic region is at least 80%, 85%, 90%, 95%, or 100% identical to amino acids 289-474 of SEQ ID NO: 2. In some embodiments, the genome of the animal comprises a sequence that is at least 80%, 85%, 90%, 95%, or 100% identical to SEQ ID NO: 7.

In some embodiments, the present disclosure also provides a chimeric (e.g., humanized) TNFR2 nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse TNFR2 mRNA sequence (e.g., SEQ ID NO: 1), mouse TNFR2 amino acid sequence (e.g., SEQ ID NO: 2), or a portion thereof (e.g., a portion of exon 2, exon 3, exon 4, exon 5, and a portion of exon 6); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human TNFR2 mRNA sequence (e.g., SEQ ID NO: 3), human TNFR2 amino acid sequence (e.g., SEQ ID NO: 4), or a portion thereof (e.g., a portion of exon 2, exon 3, exon 4, exon 5, and a portion of exon 6).

In some embodiments, the sequence encoding amino acids 33-260 of mouse TNFR2 (SEQ ID NO: 2) is replaced. In some embodiments, the sequence is replaced by a sequence encoding a corresponding region of human TNFR2 (e.g., amino acids 33-259 of human TNFR2 (SEQ ID NO: 4)).

In some embodiments, the sequence encoding amino acids 23-258 of mouse TNFR2 (SEQ ID NO: 2) is replaced. In some embodiments, the sequence is replaced by a sequence encoding a corresponding region of human TNFR2 (e.g., amino acids 23-257 of human TNFR2 (SEQ ID NO: 4)).

In some embodiments, the nucleic acids as described herein are operably linked to a promotor or regulatory element, e.g., an endogenous mouse TNFR2 promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from part of or the entire mouse TNFR2 nucleotide sequence (e.g., exon 2, exon 3, exon 4, exon 5, exon 6, or NM_011610.3 (SEQ ID NO: 1)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire mouse TNFR2 nucleotide sequence (e.g., exon 2, exon 3, exon 4, exon 5, exon 6, or NM_011610.3 (SEQ ID NO: 1)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from part of or the entire human TNFR2 nucleotide sequence (e.g., exon 2, exon 3, exon 4, exon 5, exon 6, or NM_001066.2 (SEQ ID NO: 3)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire human TNFR2 nucleotide sequence (e.g., exon 2, exon 3, exon 4, exon 5, exon 6, or NM_001066.2 (SEQ ID NO: 3)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire mouse TNFR2 amino acid sequence (e.g., amino acids encoded by exon 2, exon 3, exon 4, exon 5, and/or exon 6 of NM_011610.3 (SEQ ID NO: 1); or NP_035740.2 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire mouse TNFR2 amino acid sequence (e.g., amino acids encoded by exon 2, exon 3, exon 4, exon 5, and/or exon 6 of NM_011610.3 (SEQ ID NO: 1); or NP_035740.2 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire human TNFR2 amino acid sequence (e.g., amino acids encoded by exon 2, exon 3, exon 4, exon 5, and/or exon 6 of NM_001066.2 (SEQ ID NO: 3); or NP_001057.1 (SEQ ID NO: 4)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire human TNFR2 amino acid sequence (e.g., amino acids encoded by exon 2, exon 3, exon 4, exon 5, and/or exon 6 of NM_001066.2 (SEQ ID NO: 3); or NP_001057.1 (SEQ ID NO: 4)).

The present disclosure also provides a humanized TNFR2 mouse amino acid sequence, wherein the amino acid sequence is selected from the group consisting of:

a) an amino acid sequence shown in SEQ ID NO: 9;

b) an amino acid sequence having a homology of at least 90% with or at least 90% identical to the amino acid sequence shown in SEQ ID NO: 9;

c) an amino acid sequence encoded by a nucleic acid sequence, wherein the nucleic acid sequence is able to hybridize to a nucleotide sequence encoding the amino acid shown in SEQ ID NO: 9 under a low stringency condition or a strict stringency condition;

d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 9;

e) an amino acid sequence that is different from the amino acid sequence shown in SEQ ID NO: 9 by no more than 10, 9, 8, 7, 6, 5, 4, 3, 2 or no more than 1 amino acid; or f) an amino acid sequence that comprises a substitution, a deletion and/or insertion of one or more amino acids to the amino acid sequence shown in SEQ ID NO: 9.

The present disclosure also relates to a TNFR2 nucleic acid (e.g., DNA or RNA) sequence, wherein the nucleic acid sequence can be selected from the group consisting of:

a) a nucleic acid sequence as shown in SEQ ID NO:7, 8, 10, or 11, or a nucleic acid sequence encoding a homologous TNFR2 amino acid sequence of a humanized mouse TNFR2;

b) a nucleic acid sequence that is able to hybridize to the nucleotide sequence as shown in SEQ ID NO:7, 8, 10, or 11 under a low stringency condition or a strict stringency condition;

c) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 7, 8, 10, or 11;

d) a nucleic acid sequence that encodes an amino acid sequence, wherein the amino acid sequence has a homology of at least 90% with or at least 90% identical to the amino acid sequence shown in SEQ ID NO: 9;

e) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 9;

f) a nucleic acid sequence that encodes an amino acid sequence, wherein the amino acid sequence is different from the amino acid sequence shown in SEQ ID NO: 9 by no more than 10, 9, 8, 7, 6, 5, 4, 3, 2 or no more than 1 amino acid; and/or

g) a nucleic acid sequence that encodes an amino acid sequence, wherein the amino acid sequence comprises a substitution, a deletion and/or insertion of one or more amino acids to the amino acid sequence shown in SEQ ID NO: 9.

The present disclosure further relates to a TNFR2 genomic DNA sequence of a humanized mouse. The DNA sequence is obtained by reverse transcription of the mRNA obtained by transcription thereof is consistent with or complementary to the DNA sequence homologous to the sequence shown in SEQ ID NO:8.

The disclosure also provides an amino acid sequence that has a homology of at least 90% with, or at least 90% identical to the sequence shown in SEQ ID NO: 9, and has protein activity. In some embodiments, the homology with the sequence shown in SEQ ID NO: 9 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NO: 9 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 9, and encodes a polypeptide that has protein activity. In some embodiments, the homology with the sequence shown in SEQ ID NO: 9 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

In some embodiments, the percentage identity with the sequence shown in SEQ ID NO: 7, 8, 10, or 11 is at least about 90%, 91%, 92%, 93%, 94%, 95%, 96%, 97%, 98%, or at least 99%. In some embodiments, the foregoing percentage identity is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) TNFR2 from an endogenous non-human TNFR2 locus.

### Genetically Modified Animals

As used herein, the term “genetically-modified non-human animal” refers to a non-human animal having exogenous DNA in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the exogenous DNA in its genome. The cell having exogenous DNA can be various kinds of cells, e.g., an endogenous cell, a somatic cell, an immune cell, a T cell, a B cell, an antigen presenting cell, a macrophage, a dendritic cell, a germ cell, a blastocyst, or an endogenous tumor cell. In some embodiments, genetically-modified non-human animals are provided that comprise a modified endogenous TNFR2 locus that comprises an exogenous sequence (e.g., a human sequence), e.g., a replacement of one or more non-human sequences with one or more human sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wild-type nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one of the sequences of the protein or the polypeptide does not correspond to wild-type amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

In some embodiments, the chimeric gene or the chimeric nucleic acid is a humanized TNFR2 gene or a humanized TNFR2 nucleic acid. In some embodiments, at least one or more portions of the gene or the nucleic acid is from the human TNFR2 gene, at least one or more portions of the gene or the nucleic acid is from a non-human TNFR2 gene. In some embodiments, the gene or the nucleic acid comprises a sequence that encodes an TNFR2 protein. The encoded TNFR2 protein is functional or has at least one activity of the human TNFR2 protein or the non-human TNFR2 protein, e.g., binding to TNFα; interacting with TRAF1, TRAF2, and/or TRAF3; recruiting cIAP1/2 proteins; activating NFκB pathway; promoting tumorigenesis and progression; activating extracellular signal-regulated kinase (ERK) pathway; activating myosin light-chain kinase (MLCK) pathway; activating PI3K/AKT signaling pathway; accelerating malignant transformation and growth of tumor cells; inducing angiogenesis; inducing cancer-associated fibroblast (CAF); inducing IL6 and/or IL33 secretion; promoting cell division; promoting cell (e.g., cancer cell) proliferation; promoting Treg cell development and differentiation; increase relative ratio of Teff:Treg; inducing apoptosis; promoting tissue (e.g., neural tissue) regeneration; and/or facilitating metastasis.

In some embodiments, the chimeric protein or the chimeric polypeptide is a humanized TNFR2 protein or a humanized TNFR2 polypeptide. In some embodiments, at least one or more portions of the amino acid sequence of the protein or the polypeptide is from a human TNFR2 protein, and at least one or more portions of the amino acid sequence of the protein or the polypeptide is from a non-human TNFR2 protein. The humanized TNFR2 protein or the humanized TNFR2 polypeptide is functional or has at least one activity of the human TNFR2 protein or the non-human TNFR2 protein.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. These mice are described, e.g., in Festing et al., Revised nomenclature for strain 129 mice, Mammalian Genome 10: 836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized TNFR2 animal is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD mice, SCID mice, NOD/SCID mice, IL2Rγ knockout mice, NOD/SCID/γcnull mice (Ito, M. et al., NOD/SCID/γcnull mouse: an excellent recipient mouse model for engraftment of human cells, Blood 100(9): 3175-3182, 2002), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of an endogenous non-human TNFR2 locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, IL-2Rγ knockout mice, NOD/SCID/γc null mice, nude mice, Rag1 and/or Rag2 knockout mice, and a combination thereof. These genetically modified animals are described, e.g., in US20150106961, which is incorporated herein by reference in its entirety. In some embodiments, the mouse can include a replacement of all or part of mature TNFR2 coding sequence with human mature TNFR2 coding sequence. In some embodiments, the mature TNFR2 is the soluble TNFR2 as described herein.

Genetically modified non-human animals that comprise a modification of an endogenous non-human TNFR2 locus. In some embodiments, the modification can comprise a human nucleic acid sequence encoding at least a portion of a mature TNFR2 protein (e.g., at least 10%, 20%, 30%, 40%, 50%, 60%, 70%, 80%, 90%, 95%, 96%, 97%, 98%, or 99% identical to the mature TNFR2 protein sequence). Although genetically modified cells are also provided that can comprise the modifications described herein (e.g., ES cells, somatic cells), in many embodiments, the genetically modified non-human animals comprise the modification of the endogenous TNFR2 locus in the germline of the animal.

Genetically modified animals can express a human TNFR2 and/or a chimeric (e.g., humanized) TNFR2 from endogenous mouse loci, wherein the endogenous mouse TNFR2 gene has been replaced with a human TNFR2 gene and/or a nucleotide sequence that encodes a region of human TNFR2 sequence or an amino acid sequence that is at least 10%, 20%, 30%, 40%, 50%, 60%, 70&, 80%, 90%, 95%, 96%, 97%, 98%, or 99% identical to the human TNFR2 sequence. In various embodiments, an endogenous non-human TNFR2 locus is modified in whole or in part to comprise human nucleic acid sequence encoding at least one protein-coding sequence of a mature TNFR2 protein.

In some embodiments, the genetically modified mice express the human TNFR2 and/or chimeric TNFR2 (e.g., humanized TNFR2) from endogenous loci that are under control of mouse promoters and/or mouse regulatory elements. The replacement(s) at the endogenous mouse loci provide non-human animals that express human TNFR2 or chimeric TNFR2 (e.g., humanized TNFR2) in appropriate cell types and in a manner that does not result in the potential pathologies observed in some other transgenic mice known in the art. The human TNFR2 or the chimeric TNFR2 (e.g., humanized TNFR2) expressed in animal can maintain one or more functions of the wild-type mouse or human TNFR2 in the animal. For example, human or non-human TNFR2 ligands (e.g., TNFα) can bind to the expressed TNFR2, which further activates NF-κB signaling pathway, e.g., increase cell survival rate by at least 10%, 20%, 30%, 40%, or 50%. Furthermore, in some embodiments, the animal does not express endogenous TNFR2. In some embodiments, the animal expresses a decreased level of endogenous TNFR2 as compared to a wild-type animal. As used herein, the term “endogenous TNFR2” refers to TNFR2 protein that is expressed from an endogenous TNFR2 nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human TNFR2 (NP_001057.1) (SEQ ID NO: 4). In some embodiments, the genome comprises a sequence encoding an amino acid sequence that is at least 70%, 75%, 80%, 85%, 90%, 95%, 99%, or 100% identical to SEQ ID NO: 9.

The genome of the genetically modified animal can comprise a replacement at an endogenous TNFR2 gene locus of a sequence encoding a region of endogenous TNFR2 with a sequence encoding a corresponding region of human TNFR2. In some embodiments, the sequence that is replaced is any sequence within the endogenous TNFR2 gene locus, e.g., exon 1, exon 2, exon 3, exon 4, exon 5, exon 6, exon 7, exon 8, exon 9, exon 10, 5′-UTR, 3′-UTR, the first intron, the second intron, and the third intron, the fourth intron, the fifth intron, the sixth intron, the seventh intron, the eighth intron, the ninth intron, etc. In some embodiments, the sequence that is replaced is within the regulatory region of the endogenous TNFR2 gene. In some embodiments, the sequence that is replaced is exon 2, exon 3, exon 4, exon 5, exon 6, or a portion thereof, of an endogenous mouse TNFR2 gene locus.

The genetically modified animal can have one or more cells expressing a human or chimeric TNFR2 (e.g., humanized TNFR2) having an extracellular region and a cytoplasmic region, wherein the extracellular region comprises a sequence that is at least 50%, 60%, 70%, 80%, 90%, 95%, 99% identical to the extracellular region of human TNFR2. In some embodiments, the extracellular region of the humanized TNFR2 has a sequence that has at least 10, 20, 30, 40, 50, 60, 70, 80, 90, 100, 110, 120, 130, 140, 150, 160, 170, or 180 amino acids (e.g., contiguously or non-contiguously) that are identical to human TNFR2. Because human TNFR2 and non-human TNFR2 (e.g., mouse TNFR2) sequences, in many cases, are different, antibodies that bind to human TNFR2 will not necessarily have the same binding affinity with non-human TNFR2 or have the same effects to non-human TNFR2. Therefore, the genetically modified animal having a human or a humanized extracellular region can be used to better evaluate the effects of anti-human TNFR2 antibodies in an animal model. In some embodiments, the genome of the genetically modified animal comprises a sequence encoding an amino acid sequence that corresponds to a portion or the entire sequence of exon 2, exon 3, exon 4, exon 5, and/or exon 6 of human TNFR2, a portion or the entire sequence of extracellular region of human TNFR2 (with or without signal peptide), or a portion or the entire sequence of amino acids 23-257, or 33-259 of SEQ ID NO: 4.

In some embodiments, the non-human animal can have, at an endogenous TNFR2 gene locus, a nucleotide sequence encoding a chimeric human/non-human TNFR2 polypeptide, wherein a human portion of the chimeric human/non-human TNFR2 polypeptide comprises a portion of human TNFR2 extracellular domain, and wherein the animal expresses a functional TNFR2 on a surface of a cell of the animal. The human portion of the chimeric human/non-human TNFR2 polypeptide can comprise an amino acid sequence encoded by a portion of exon 2, exon 3, exon 4, exon 5, and/or exon 6 of human TNFR2. In some embodiments, the human portion of the chimeric human/non-human TNFR2 polypeptide can comprise a sequence that is at least 80%, 85%, 90%, 95%, or 99% identical to amino acids 23-257, or 33-259 of SEQ ID NO: 4. In some embodiments, the human portion of the chimeric human/non-human TNFR2 polypeptide comprises at least or about 1, 2, 3, 4, 5, 6, 7, or 8 amino acids of human TNFR2 transmembrane region. In some embodiments, the transmembrane region includes a sequence corresponding to the entire or part of amino acids 259-288 of SEQ ID NO: 2. In some embodiments, the transmembrane region includes a sequence corresponding to the entire of part of amino acids 258-287 of SEQ ID NO: 4.

In some embodiments, the non-human portion of the chimeric human/non-human TNFR2 polypeptide comprises transmembrane and/or cytoplasmic regions of an endogenous non-human TNFR2 polypeptide.

Furthermore, the genetically modified animal can be heterozygous with respect to the replacement at the endogenous TNFR2 locus, or homozygous with respect to the replacement at the endogenous TNFR2 locus.

In some embodiments, the humanized TNFR2 locus lacks a human TNFR2 5′-UTR. In some embodiment, the humanized TNFR2 locus comprises an endogenous (e.g., mouse) 5′-UTR. In some embodiments, the humanization comprises an endogenous (e.g., mouse) 3′-UTR. In appropriate cases, it may be reasonable to presume that the mouse and human TNFR2 genes appear to be similarly regulated based on the similarity of their 5′-flanking sequence. As shown in the present disclosure, humanized TNFR2 mice that comprise a replacement at an endogenous mouse TNFR2 locus, which retain mouse regulatory elements but comprise a humanization of TNFR2 encoding sequence, do not exhibit pathologies. Both genetically modified mice that are heterozygous or homozygous for humanized TNFR2 are grossly normal.

The present disclosure further relates to a non-human mammal generated through the method mentioned above. In some embodiments, the genome thereof contains human gene(s).

In some embodiments, the non-human mammal is a rodent, and preferably, the non-human mammal is a mouse.

In some embodiments, the non-human mammal expresses a protein encoded by a humanized TNFR2 gene.

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains the DNA encoding human or humanized TNFR2 in the genome of the animal.

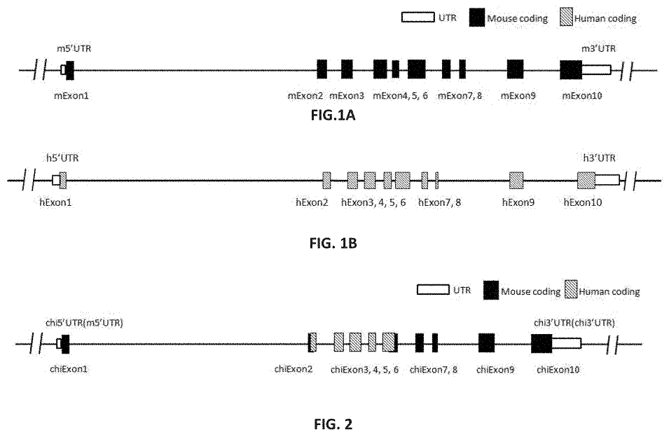

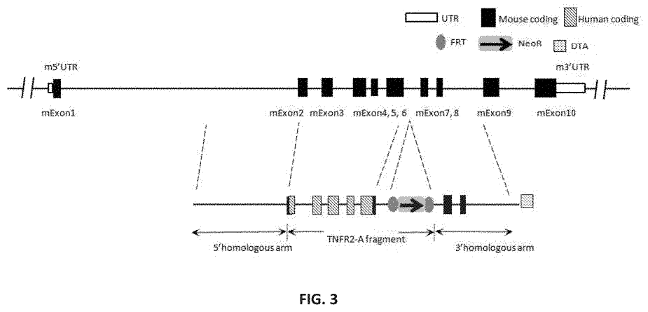

In some embodiments, the non-human mammal comprises the genetic construct as described herein (e.g., gene construct as shown in FIG. 2 or FIG. 3). In some embodiments, a non-human mammal expressing human or humanized TNFR2 is provided. In some embodiments, the tissue-specific expression of human or humanized TNFR2 protein is provided.

In some embodiments, the expression of human or humanized TNFR2 in a genetically modified animal is controllable, as by the addition of a specific inducer or repressor substance. In some embodiments, the specific inducer is selected from Tet-Off System/Tet-On System, or Tamoxifen System.

Non-human mammals can be any non-human animal known in the art and which can be used in the methods as described herein. Preferred non-human mammals are mammals, (e.g., rodents). In some embodiments, the non-human mammal is a mouse.

Genetic, molecular and behavioral analyses for the non-human mammals described above can performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cells can be obtained from the cell culture established using the same constructs and the standard cell transfection techniques. The integration of genetic constructs containing DNA sequences encoding human TNFR2 protein can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized TNFR2 protein.

### Vectors

The present disclosure relates to a targeting vector, comprising: a) a DNA fragment homologous to the 5′ end of a region to be altered (5′ arm), which is selected from the TNFR2 gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the TNFR2 gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000070.6; c) the DNA fragment homologous to the 3′ end of the region to be altered (3′ arm) is selected from the nucleotide sequences that have at least 90% homology to the NCBI accession number NC_000070.6.

In some embodiments, a) the DNA fragment homologous to the 5′ end of a region to be altered (5′ arm) is selected from the nucleotides from the position 145233556 to the position 145229089 of the NCBI accession number NC_000070.6; c) the DNA fragment homologous to the 3′ end of the region to be altered (3′ arm) is selected from the nucleotides from the position 145223912 to the position 145220426 of the NCBI accession number NC_000070.6.

In some embodiments, the length of the selected genomic nucleotide sequence in the targeting vector can be more than about 3 kb, about 3.5 kb, about 4 kb, about 4.5 kb, about 5 kb, about 5.5 kb, about 6 kb, about 6.5 kb, or about 7 kb.

In some embodiments, the region to be altered is exon 1, exon 2, exon 3, exon 4, exon 5, exon 6, exon 7, exon 8, exon 9, and/or exon 10 of TNFR2 gene (e.g., exon 2, exon 3, exon 4, exon 5, and/or exon 6 of mouse TNFR2 gene).

The targeting vector can further include one or more selectable markers, e.g., positive or negative selectable markers. In some embodiments, the positive selectable marker is a Neo gene or Neo cassette. In some embodiments, the negative selectable marker is a DTA gene.

In some embodiments, the sequence of the 5′ arm is shown in SEQ ID NO: 5; and the sequence of the 3′ arm is shown in SEQ ID NO: 6.

In some embodiments, the sequence is derived from human (e.g., 12188814-12193088 of NC_000001.11). For example, the target region in the targeting vector is a part or entirety of the nucleotide sequence of a human TNFR2, preferably exon 2, exon 3, exon 4, exon 5, and/or exon 6 of the human TNFR2. In some embodiments, the nucleotide sequence of the humanized TNFR2 encodes the entire or the part of human TNFR2 protein with the NCBI accession number NP_001057.1 (SEQ ID NO: 4).

The disclosure also relates to a cell comprising the targeting vectors as described above. In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous TNFR2 gene locus, a sequence encoding a region of an endogenous TNFR2 with a sequence encoding a corresponding region of human or chimeric TNFR2. In some embodiments, the replacement occurs in a germ cell, a somatic cell, a blastocyst, or a fibroblast, etc. The nucleus of a somatic cell or the fibroblast can be inserted into an enucleated oocyte.

FIG. 3 shows a humanization strategy for a mouse TNFR2 locus. In FIG. 3, the targeting strategy involves a vector comprising the 5′ end homologous arm, human TNFR2 gene fragment, 3′ homologous arm. The process can involve replacing endogenous TNFR2 sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous TNFR2 sequence with human TNFR2 sequence.

Thus, in some embodiments, the methods for making a genetically modified, humanized animal, can include the step of replacing at an endogenous TNFR2 locus (or site), a nucleic acid encoding a sequence encoding a region of endogenous TNFR2 with a sequence encoding a corresponding region of human TNFR2. The sequence can include a region (e.g., a part or the entire region) of exon 1, exon 2, exon 3, exon 4, exon 5, exon 6, exon 7, exon 8, exon 9, and/or exon 10 of a human TNFR2 gene. In some embodiments, the sequence includes a region of exon 2, exon 3, exon 4, exon 5, and exon 6 of a human TNFR2 gene (e.g., amino acids 33-259 of SEQ ID NO: 4). In some embodiments, the sequence includes a region of exon 1, exon 2, exon 3, exon 4, exon 5, and exon 6 of a human TNFR2 gene (e.g., amino acids 23-257 of SEQ ID NO: 4). In some embodiments, the region is located within the extracellular region and/or transmembrane region of TNFR2 (e.g., amino acids 23-257, or 33-259 of SEQ ID NO: 4). In some embodiments, the endogenous TNFR2 locus is exon 1, exon 2, exon 3, exon 4, exon 5, and/or exon 6 of mouse TNFR2.

In some embodiments, the methods of modifying a TNFR2 locus of a mouse to express a chimeric human/mouse TNFR2 peptide can include the steps of replacing at the endogenous mouse TNFR2 locus a nucleotide sequence encoding a mouse TNFR2 with a nucleotide sequence encoding a human TNFR2, thereby generating a sequence encoding a chimeric human/mouse TNFR2.

In some embodiments, the nucleotide sequence encoding the chimeric human/mouse TNFR2 can include a first nucleotide sequence encoding an extracellular region of mouse TNFR2 (with or without the mouse or human signal peptide sequence); a second nucleotide sequence encoding an extracellular region of human TNFR2; a third nucleotide sequence encoding a transmembrane and a cytoplasmic region of a mouse TNFR2.

In some embodiments, the nucleotide sequences as described herein do not overlap with each other (e.g., the first nucleotide sequence, the second nucleotide sequence, and/or the third nucleotide sequence do not overlap). In some embodiments, the amino acid sequences as described herein do not overlap with each other.

The present disclosure further provides a method for establishing a TNFR2 gene humanized animal model, involving the following steps:

(a) providing the cell (e.g. a fertilized egg cell) based on the methods described herein;

(b) culturing the cell in a liquid culture medium;

(c) transplanting the cultured cell to the fallopian tube or uterus of the recipient female non-human mammal, allowing the cell to develop in the uterus of the female non-human mammal;

(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudopregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

### Methods of Using Genetically Modified Animals

Replacement of non-human genes in a non-human animal with homologous or orthologous human genes or human sequences, at the endogenous non-human locus and under control of endogenous promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal's genome and presumably integrates at random into the genome. Typically, the location of the integrated transgene is unknown; expression of the human protein is measured by transcription of the human gene and/or protein assay and/or functional assay. Inclusion in the human transgene of upstream and/or downstream human sequences are apparently presumed to be sufficient to provide suitable support for expression and/or regulation of the transgene.

In some cases, the transgene with human regulatory elements expresses in a manner that is unphysiological or otherwise unsatisfactory, and can be actually detrimental to the animal. The disclosure demonstrates that a replacement with human sequence at an endogenous locus under control of endogenous regulatory elements provides a physiologically appropriate expression pattern and level that results in a useful humanized animal whose physiology with respect to the replaced gene are meaningful and appropriate in the context of the humanized animal's physiology.

Genetically modified animals that express human or humanized TNFR2 protein, e.g., in a physiologically appropriate manner, provide a variety of uses that include, but are not limited to, developing therapeutics for human diseases and disorders, and assessing the toxicity and/or the efficacy of these human therapeutics in the animal models.

In various aspects, genetically modified animals are provided that express human or humanized TNFR2, which are useful for testing agents that can decrease or block the interaction between TNFR2 and TNFR2 ligands (e.g., TNFα) or the interaction between TNFR2 and anti-human TNFR2 antibodies, testing whether an agent can increase or decrease the immune response, and/or determining whether an agent is an TNFR2 agonist or antagonist. The genetically modified animals can be, e.g., an animal model of a human disease, e.g., the disease is induced genetically (a knock-in or knockout). In various embodiments, the genetically modified non-human animals further comprise an impaired immune system, e.g., a non-human animal genetically modified to sustain or maintain a human xenograft, e.g., a human solid tumor or a blood cell tumor (e.g., a lymphocyte tumor, e.g., a B or T cell tumor. In some embodiments, the anti-TNFR2 antibody blocks or inhibits the TNFR2-related signaling pathway.

In some embodiments, the genetically modified animals can be used for determining effectiveness of an anti-TNFR2 antibody for the treatment of cancer. The methods involve administering the anti-TNFR2 antibody (e.g., anti-human TNFR2 antibody) to the animal as described herein, wherein the animal has a tumor; and determining the inhibitory effects of the anti-TNFR2 antibody to the tumor. The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MM or CT. Without wishing to be bound by a particular theory, it is believed that in some embodiments, these anti-TNFR2 antibodies can target regulatory T cells (Treg) and kill or inhibit the function of Treg cells, as TNFR2 is highly expressed on Treg cells, thereby increasing the immune response. In addition, a delicate balance is required for these antibodies, as TNFR2 is also expressed on many other cells. Thus, it is important that the humanized TNFR2 functions in a largely similar way as compared to the endogenous TNFR2, so that the results in the humanized animals can be used to predict the efficacy or toxicity of these therapeutic agents in the human. In some embodiments, the anti-TNFR2 antibody can directly target cancer cells expressing TNFR2, e.g., by inducing complement mediated cytotoxicity (CMC) or antibody dependent cellular cytoxicity (ADCC) to kill the cancer cells.

In some embodiments, the tumor comprises one or more cancer cells (e.g., human or mouse cancer cells) that are injected into the animal. In some embodiments, the anti-TNFR2 antibody, or anti-TNFα antibody prevents TNFα from binding to TNFR2. In some embodiments, the anti-TNFR2 antibody or anti-TNFα antibody does not prevent TNFα from binding to TNFR2.

In some embodiments, the genetically modified animals can be used for determining whether an anti-TNFR2 antibody is a TNFR2 agonist or antagonist. In some embodiments, the methods as described herein are also designed to determine the effects of the agent (e.g., anti-TNFR2 antibodies) on TNFR2, e.g., whether the agent can stimulate immune cells or inhibit immune cells (e.g., T cells, B cells, or NK cells), whether the agent can increase or decrease the production of cytokines, whether the agent can activate or deactivate immune cells (e.g., T cells, B cells, or NK cells), whether the agent can upregulate the immune response or downregulate immune response, and/or whether the agent can induce complement mediated cytotoxicity (CMC) or antibody dependent cellular cytoxicity (ADCC). In some embodiments, the genetically modified animals can be used for determining the effective dosage of a therapeutic agent for treating a disease in the subject, e.g., cancer, or autoimmune diseases.

The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV} (%)=(1−TVt/TVc)×100, where TVt and TVc are the mean tumor volume (or weight) of treated and control groups.

In some embodiments, the anti-TNFR2 antibody is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the anti-TNFR2 antibody is designed for treating melanoma (e.g., advanced melanoma), non-small cell lung carcinoma (NSCLC), small cell lung cancer (SCLC), B-cell non-Hodgkin lymphoma, bladder cancer, and/or prostate cancer (e.g., metastatic hormone-refractory prostate cancer). In some embodiments, the anti-TNFR2 antibody is designed for treating hepatocellular, ovarian, colon, or cervical carcinomas. In some embodiments, the anti-TNFR2 antibody is designed for treating advanced breast cancer, advanced ovarian cancer, and/or advanced refractory solid tumor. In some embodiments, the anti-TNFR2 antibody is designed for treating metastatic solid tumors, NSCLC, melanoma, non-Hodgkin lymphoma, colorectal cancer, and multiple myeloma. In some embodiments, the anti-TNFR2 antibody is designed for treating melanoma, pancreatic carcinoma, mesothelioma, hematological malignancies (e.g., Non-Hodgkin's lymphoma, lymphoma, chronic lymphocytic leukemia), or solid tumors (e.g., advanced solid tumors). In some embodiments, the anti-TNFR2 antibody is designed for treating carcinomas (e.g., nasopharynx carcinoma, bladder carcinoma, cervix carcinoma, kidney carcinoma or ovary carcinoma).

In some embodiments, the TNFR2 antibody is designed for treating breast cancer, colon cancer, cervical cancer, fibrosarcoma, liver cancer, lung cancer, non-small cell lung cancer (NSCLC), melanoma, ovarian cancer, renal cancer, skin cancer, plasmacytoma, lymphoma, and/or leukemia.

In some embodiments, the anti-TNFR2 antibody is designed for treating various autoimmune diseases, including rheumatoid arthritis, Crohn's disease, systemic lupus erythematosus, ankylosing spondylitis, inflammatory bowel diseases (IBD), ulcerative colitis, or scleroderma. In some embodiments, the anti-TNFR2 antibody is designed for treating various immune disorders, including allergy, asthma, and/or atopic dermatitis. Thus, the methods as described herein can be used to determine the effectiveness of an anti-TNFR2 antibody in inhibiting immune response.

The present disclosure also provides methods of determining toxicity of an antibody (e.g., anti-TNFR2 antibody). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%. In some embodiments, the animals can have a weight that is at least 5%, 10%, 20%, 30%, or 40% smaller than the weight of the control group (e.g., average weight of the animals that are not treated with the antibody).

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the TNFR2 gene function, human TNFR2 antibodies, drugs for human TNFR2 targeting sites, the drugs or efficacies for human TNFR2 targeting sites, the drugs for immune-related diseases and antitumor drugs.

In some embodiments, the disclosure provides a method to verify in vivo efficacy of TCR-T, CAR-T, and/or other immunotherapies (e.g., T-cell adoptive transfer therapies). For example, the methods include transplanting human tumor cells into the animal described herein, and applying human CAR-T to the animal with human tumor cells. Effectiveness of the CAR-T therapy can be determined and evaluated. In some embodiments, the animal is selected from the TNFR2 gene humanized non-human animal prepared by the methods described herein, the TNFR2 gene humanized non-human animal described herein, the double- or multi-humanized non-human animal generated by the methods described herein (or progeny thereof), a non-human animal expressing the human or humanized TNFR2 protein, or the tumor-bearing or inflammatory animal models described herein. In some embodiments, the TCR-T, CAR-T, and/or other immunotherapies can treat the TNFR2-associated diseases described herein. In some embodiments, the TCA-T, CAR-T, and/or other immunotherapies provides an evaluation method for treating the TNFR2-associated diseases described herein.

Genetically Modified Animal Model with Two or More Human or Chimeric Genes

The present disclosure further relates to methods for generating genetically modified animal model with two or more human or chimeric genes. The animal can comprise a human or chimeric TNFR2 gene and a sequence encoding an additional human or chimeric protein.

In some embodiments, the additional human or chimeric protein can be tumor necrosis factor alpha (TNFα), programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (TNFRSF4 or OX40).

The methods of generating genetically modified animal model with two or more human or chimeric genes (e.g., humanized genes) can include the following steps:

(a) using the methods of introducing human TNFR2 gene or chimeric TNFR2 gene as described herein to obtain a genetically modified non-human animal;

(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric TNFα, PD-1, CTLA-4, LAG-3, BTLA, PD-L1, CD27, CD28, CD47, CD137, CD154, TIGIT, TIM-3, GITR, SIRPα, or OX40. Some of these genetically modified non-human animal are described, e.g., in PCT/CN2017/090320, PCT/CN2017/099577, PCT/CN2017/099575, PCT/CN2017/099576, PCT/CN2017/099574, PCT/CN2017/106024, PCT/CN2017/110494, PCT/CN2017/110435, PCT/CN2017/120388, PCT/CN2018/081628, PCT/CN2018/081629; each of which is incorporated herein by reference in its entirety.

In some embodiments, the TNFR2 humanization is directly performed on a genetically modified animal having a human or chimeric TNFα, PD-1, CTLA-4, BTLA, PD-L1, CD27, CD28, CD47, CD137, CD154, TIGIT, TIM-3, GITR, SIRPα, or OX40 gene.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., an anti-TNFR2 antibody and an additional therapeutic agent for the treatment of cancer. The methods include administering the anti-TNFR2 antibody and the additional therapeutic agent to the animal, wherein the animal has a tumor; and determining the inhibitory effects of the combined treatment to the tumor. In some embodiments, the additional therapeutic agent is an antibody that specifically binds to TNFα, CD122, CD132, PD-1, CTLA-4, BTLA, PD-L1, CD27, CD28, CD47, CD137, CD154, TIGIT, TIM-3, GITR, SIRPα, or OX40. In some embodiments, the additional therapeutic agent is an anti-CTLA4 antibody (e.g., ipilimumab), an anti-PD-1 antibody (e.g., nivolumab), or an anti-PD-L1 antibody.

In some embodiments, the animal further comprises a sequence encoding a human or humanized PD-1, a sequence encoding a human or humanized PD-L1, or a sequence encoding a human or humanized CTLA-4. In some embodiments, the additional therapeutic agent is an anti-PD-1 antibody (e.g., nivolumab, pembrolizumab), an anti-PD-L1 antibody, or an anti-CTLA-4 antibody. In some embodiments, the tumor comprises one or more tumor cells that express CD80, CD86, PD-L1, and/or PD-L2.

In some embodiments, the combination treatment is designed for treating various cancer as described herein, e.g., melanoma, non-small cell lung carcinoma (NSCLC), small cell lung cancer (SCLC), bladder cancer, prostate cancer (e.g., metastatic hormone-refractory prostate cancer), advanced breast cancer, advanced ovarian cancer, and/or advanced refractory solid tumor. In some embodiments, the combination treatment is designed for treating metastatic solid tumors, NSCLC, melanoma, B-cell non-Hodgkin lymphoma, colorectal cancer, and multiple myeloma. In some embodiments, the combination treatment is designed for treating melanoma, carcinomas (e.g., pancreatic carcinoma), mesothelioma, hematological malignancies (e.g., Non-Hodgkin's lymphoma, lymphoma, chronic lymphocytic leukemia), or solid tumors (e.g., advanced solid tumors). In some embodiments, the combination treatment is designed for treating breast cancer, colon cancer, cervical cancer, fibrosarcoma, liver cancer, lung cancer, non-small cell lung cancer (NSCLC), melanoma, ovarian cancer, renal cancer, skin cancer, plasmacytoma, lymphoma, and/or leukemia.

In some embodiments, the methods described herein can be used to evaluate the combination treatment with some other methods. The methods of treating a cancer that can be used alone or in combination with methods described herein, include, e.g., treating the subject with chemotherapy, e.g., campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, and/or methotrexate. Alternatively or in addition, the methods can include performing surgery on the subject to remove at least a portion of the cancer, e.g., to remove a portion of or all of a tumor(s), from the patient.

### EXAMPLES

The invention is further described in the following examples, which do not limit the scope of the invention described in the claims.

### Materials and Methods

The following materials were used in the following examples.

EcoRI, NdeI, and SspI restriction enzymes were purchased from NEB (Catalog numbers: R3101M, R0111L, and R3132M, respectively).

C57BL/6 mice and Flp mice were purchased from the China Food and Drugs Research Institute National Rodent Experimental Animal Center.

Flow cytometer was purchased from BD Biosciences (model: FACS Calibur™).

Cas9 mRNA was purchased from SIGMA (Catalog number: CAS9MRNA-1EA).

UCA kit was obtained from Beijing Biocytogen Co., Ltd. (Catalog number: BCG-DX-001).

Ambion in vitro transcription kit MEGAshortscript™ Kit was purchased from ThermoFisher Scientific (Catalog number: AM1354).

PerCP/Cyanine5.5 anti-mouse TCRβ chain (anti-mTcRβPerCP/Cy5.5) was purchased from BioLegend, Inc. (Catalog number: 109228).

FITC anti-mouse CD19 antibody (anti-mCD19 FITC) was purchased from BioLegend, Inc. (Catalog number: 115506).

PE anti-mouse CD120b (TNF R Type II/p75) antibody (anti-mTNFR2 PE) was purchased from BioLegend, Inc. (Catalog number: 113405).

PE anti-human CD120b Antibody (anti-hTNFR2 PE) was purchased from BioLegend Inc. (Catalog number: 358403).

PE Syrian Hamster IgG Isotype Ctrl Antibody was purchased from BioLegend, Inc. (Catalog number: 402008).

PE Rat IgG2a, κ Isotype Ctrl Antibody was purchased from BioLegend, Inc. (Catalog number: 400508).

APC anti-mouse/rat Foxp3 (anti-mFoxp3-APC) was purchased from eBioscience, Inc. (Catalog number: 17-5773-82).

Brilliant Violet 421™ anti-mouse CD4 (anti-mCD4-BV421) was purchased from BioLegend, Inc. (Catalog number: 100438).

RNAprep pure Kit (For Cell Bacteria) was purchased from TIANGEN Biotech (Beijing) Co., Ltd. (Catalog number: DP430).

PowerUp™ SYBR™ Green Master Mix kit was purchased from Thermo Fisher Scientific (Catalog number: A25742).

### Example 1: Mice with Humanized TNFR2 Gene

The mouse TNFR2 gene (NCBI Gene ID: 21938, Primary source: MGI: 1314883, UniProt ID: P25119) is located at 145212368 to 145246870 of chromosome 4 (NC_000070.6), and the human TNFR2 gene (NCBI Gene ID: 7133, Primary source: HGNC:11917, UniProt ID: P20333) is located at 12166948 to 12209222 of chromosome 1 (NC_000001.11). FIG. 1A shows the mouse TNFR2 gene (the transcript NM_011610.3 (SEQ ID NO: 1) and the corresponding protein sequence NP_035740.2 (SEQ ID NO: 2)). FIG. 1B shows the human TNFR2 gene (the transcript NM_001066.2 (SEQ ID NO: 3) and the corresponding protein sequence NP_001057.1 (SEQ ID NO: 4)).

A gene sequence encoding the human TNFR2 protein can be introduced into the endogenous mouse TNFR2 locus, such that the mouse can express a human or humanized TNFR2 protein. Mouse cells can be modified by various gene-editing techniques, for example, replacement of specific mouse TNFR2 gene sequences with human TNFR2 gene sequences at the endogenous mouse TNFR2 locus. For example, under control of a mouse TNFR2 regulatory element, a sequence about 8807 bp spanning from exon 2 (including only a part of exon 2) to exon 6 (including only a part of exon 6) of mouse TNFR2 gene was replaced with a corresponding human DNA sequence to obtain a humanized TNFR2 locus, thereby humanizing mouse TNFR2 gene (shown in FIG. 2).

As shown in the schematic diagram of the targeting strategy in FIG. 3, the targeting vector contained homologous arm sequences upstream and downstream of mouse TNFR2 gene locus and an “TNFR2-A fragment” comprising a human TNFR2 gene sequence. The upstream homologous arm includes about 4468 bp upstream of endogenous exon 2 and part of endogenous exon 2. The downstream homologous arm includes about 3487 bp downstream of exon 8, exons 7 and 8 of endogenous TNFR2 gene. Specifically, the upstream homologous arm sequence (5′ homologous arm, SEQ ID NO: 5) is identical to nucleotide sequence of 145233556-145229089 of NCBI accession number NC_000070.6, and the downstream homologous arm sequence (3′ homologous arm, SEQ ID NO: 6) is identical to nucleotide sequence of 145223912-145220426 of NCBI accession number NC_000070.6. The TNFR2-A fragment comprises a 4275 bp sequence spanning from within exon 2 to within exon 6 of human genomic TNFR2 gene sequence (SEQ ID NO: 7), which is identical to nucleotide sequence of 12188814-12193088 of NCBI accession number NC_000001.11. The 5′ end of the human TNFR2 gene sequence was directly connected to the 5′ homologous arm. The connection between the 3′ end of the human TNFR2 gene sequence and the mouse TNFR2 gene locus was designed as 5′-

(SEQ ID NO: 29)
GGCCCCAGCCCCCCAGCTGAAGGGAGCACTGGCGACTTCGCTCTTCCAAT

TGGTAAGTCCTCAGTCTCAAGAGTGACC-3′,

wherein the last “T” of the sequence “TCGCT” is the last nucleotide of the human sequence, and the first “C” of the sequence “CTTCC” is the first nucleotide of the mouse sequence.

The mRNA sequence of the engineered mouse TNFR2 after humanization and its encoded protein sequence are shown in SEQ ID NO: 8 and SEQ ID NO: 9, respectively. Given that human TNFR2 and mouse TNFR2 have multiple isoforms or transcripts, the methods described herein can be applied to other isoforms or transcripts.

The targeting vector also included an antibiotic resistance gene for positive clone screening (neomycin phosphotransferase gene, or Neo), and two Frt recombination sites flanking the antibiotic resistance gene, that formed a Neo cassette. The Neo cassette is located between exon 6 and exon 7 of mouse TNFR2 gene. The connection between the 5′ end of the Neo cassette and the mouse TNFR2 gene locus was designed as: 5′-

(SEQ ID NO: 10)
TCCCTTTGGGCAGAACTGGGGCCTGGTCTCTGTCTTTTAGAACGGAATTC
CGAAGTTCCTATTCTCTAGA-3′,

wherein the “C” of the sequence “TAGAAC” is the last nucleotide of the mouse sequence, and the first “G” of the sequence “GGAAT” is the first nucleotide of the Neo cassette. The connection between the 3′ end of the Neo cassette with the mouse TNFR2 gene locus was designed as 5′-

(SEQ ID NO: 11)
AGGAACTTCATCAGTCAGGTACATAATGGTGGATCCCATATGAGATGCAA

GTGTAGAAGAGTTGAGTCTC-3′,

wherein the “G” of the sequence “CATATG” is the last nucleotide of the Neo cassette, and the first “A” of the sequence “AGATGC” is the first nucleotide of the mouse sequence. In addition, a coding gene with a negative selectable marker (a gene encoding diphtheria toxin A subunit (DTA)) was also inserted downstream of the 3′ homologous arm of the targeting vector.

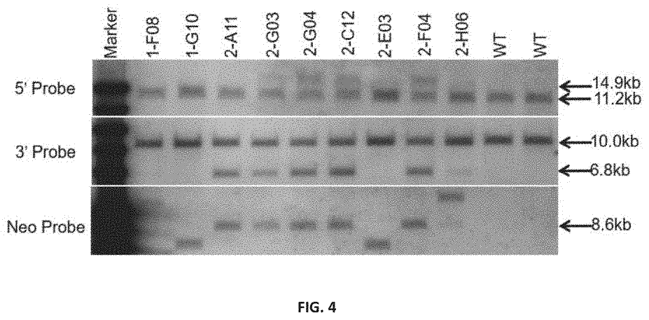

The targeting vector was constructed using standard methods, e.g., by restriction enzyme digestion and ligation. The constructed targeting vector sequence was preliminarily verified by restriction enzyme digestion, then verified by sequencing. The correct targeting vector was electroporated and transfected into embryonic stem cells of C57BL/6 mice. The positive selectable marker gene was used to screen the cells, and the integration of exogenous genes was confirmed by PCR and Southern Blot. Specifically, positive clones identified by PCR were further confirmed by Southern Blot (digested with EcoRI, NdeI, and SspI, respectively, and then hybridized with 3 corresponding probes, see Table 3) to screen out correct positive clone cells. As shown in FIG. 4, the results indicated that among the 9 positive clones confirmed by PCR, 2-G03, 2-G04, 2-C12, and 2-F04 were positive heterozygous clones and no random insertions were detected.

| | The following primers were used |
|---|---|
| | in PCR: |
| | F1: |
| (SEQ ID NO: 12) | |
| | 5′-CTCGACTGTGCCTTCTAGTTGCCAG-3′; |
| | R1: |
| (SEQ ID NO: 13) | |
| | 5′-CCTAACCTCTCTTGGTGCTGAGAAC-3′; |
| | F2: |
| (SEQ ID NO: 14) | |
| | 5′-GATCAGTGAGACAGTCCAACTTGGC-3′; |
| | R2: |
| (SEQ ID NO: 15) | |
| | 5′-GCATGGGCCAGTGCATAGAACTAG-3′. |
| | The following probes were used |
| | in Southern Blot assays: |
| | 5′ probe: |
| | 5-F: |
| (SEQ ID NO: 16) | |
| | 5′-TGATGGTGGGATGAGTCTGAAGAAG-3′, |
| | 5-R: |
| (SEQ ID NO: 17) | |
| | 5′-GAATGCCTCACCCTCTCTGCTATTA-3′; |
| | 3′ probe: |
| | 3-F: |
| (SEQ ID NO: 18) | |
| | 5′-ACCTCGAGTCAGACTTCTGTAGGTA-3′, |
| | 3-R: |
| (SEQ ID NO: 19) | |
| | 5′-CTAGGGATATAAGCAGAACGTGGCT-3′; |
| | Neo probe: |
| | Neo-F: |
| (SEQ ID NO: 20) | |
| | 5′-GGATCGGCCATTGAACAAGATGG-3′, |
| | Neo-R: |
| (SEQ ID NO: 21) | |
| | 5′-CAGAAGAACTCGTCAAGAAGGCG-3′. |

TABLE 3
Probes and respective targeted fragment size

| Restriction enzyme | Probe | WT size | Targeted size |
|---|---|---|---|
| EcoRI | 5′Probe | 11.2 kb | 14.9 kb |
| Ndel | 3′Probe | 10.0 kb | 6.8 kb |
| EcoRI | Neo Probe | — | 8.6 kb |

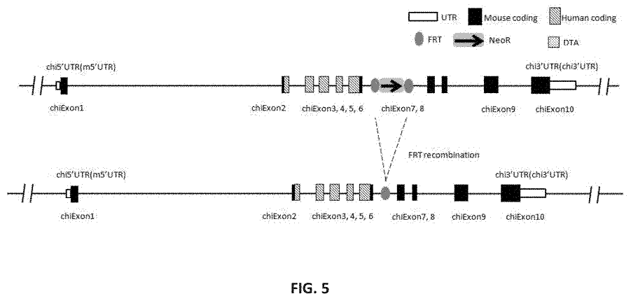

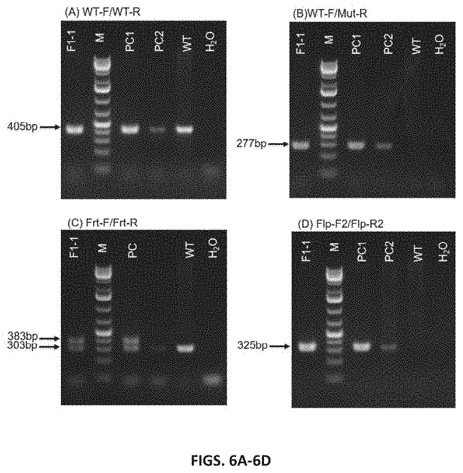

The positive clones that had been screened (black mice) were introduced into isolated blastocysts (white mice), and the resulted chimeric blastocysts were transferred to a culture medium for short-term culture and then transplanted to the fallopian tubes of the recipient mother (white mice) to produce the F0 chimeric mice (black and white). The F2 generation homozygous mice were obtained by backcrossing the F0 generation chimeric mice with wild-type mice to obtain the F1 generation mice, and then mating the F1 generation heterozygous mice with each other. The positive mice were also mated with the Flp mice to remove the positive selectable marker gene (FIG. 5), and then the humanized TNFR2 homozygous mice expressing humanized TNFR2 protein were obtained by mating with each other. The genotype of the progeny mice can be identified by PCR. The identification results of exemplary F1 generation mice (Neo cassette-removed) are shown in FIGS. 6A-6D, and the mouse labelled F1-1 was identified as a positive heterozygous clone. The following primers were used in the PCR identification. WT is wild-type mice and Mut is TNFR2 gene humanized mice.

TABLE 4
PCR primers and target band size

| | | Fragment |
|---|---|---|
| Primer | Sequence | size |
| WT-F | 5′-TATGCTGGAGCCCAGAGTCATACTG-3′ | WT:405 |
| | (SEQ ID NO: 22) | |
| WT-R | 5′-GGACATCATTGCAGTATTGAGGAGG-3′ | |
| | (SEQ ID NO: 23) | |
| WT-F | 5′-TATGCTGGAGCCCAGAGTCATACTG-3′ | Mut:277 |
| | (SEQ ID NO: 22) | |
| Mut-R | 5′-GCATGGGCCAGTGCATAGAACTAG-3′ | |
| | (SEQ ID NO: 24) | |
| Frt-F | 5′-TACTTTTAATGGGAGCTGAGGCTGT-3′ | WT:303 |
| | (SEQ ID NO: 25) | Mut:383 |
| Frt-R | 5′-ATAGTCTGTGAGACAAACGGGTTCA-3′ | |
| | (SEQ ID NO: 26) | |
| Flp-F2 | 5′-GACAAGCGTTAGTAGGCACATATAC-3′ | Mut:325b |
| | (SEQ ID NO: 27) | |
| Flp-R2 | 5′-GCTCCAATTTCCCACAACATTAGT-3′ | |
| | (SEQ ID NO: 28) | |

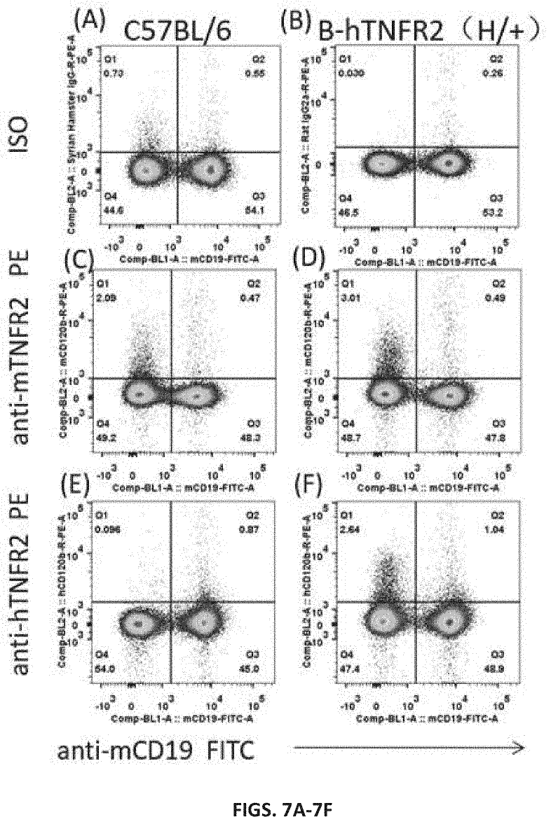

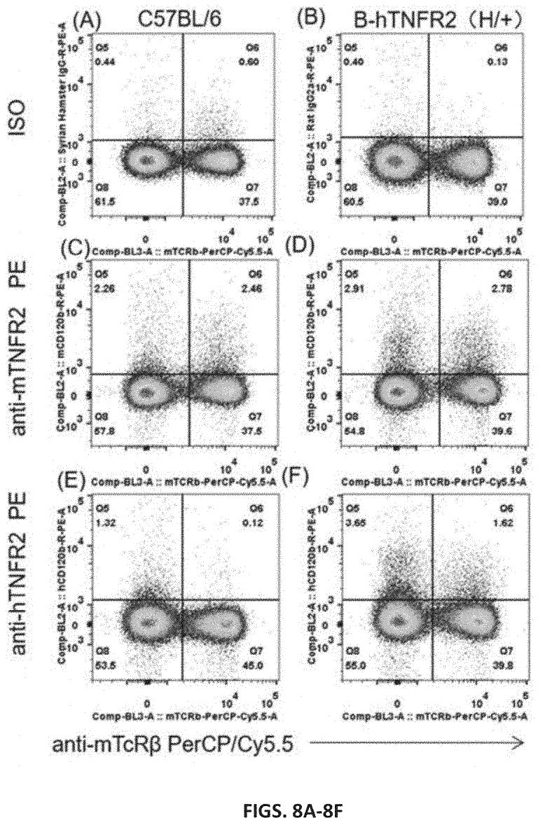

The results indicated that this method can be used to construct genetically engineered TNFR2 mice and the genetic modification can be stably passed to the next generation without random insertions. The expression of humanized TNFR2 protein in mice was confirmed by standard experiments, e.g., fluorescence-activated cell sorting (FACS). Spleen cells from 4-week old wild-type C57BL/6 mice and TNFR2 gene humanized heterozygous mice were isolated and divided into 3 groups. Each group was randomly stained using one of the following methods. First, anti-mTcRβPerCP/Cy5.5 and anti-mCD19 FITC were used to label cells. Then, the cells were stained by anti-mTNFR2 PE or anti-hTNFR2 PE, followed by flow cytometry analysis. Alternatively, isotype control antibodies (ISO) including PE Syrian Hamster IgG Isotype Ctrl Antibody and PE Rat IgG2a, κ Isotype Ctrl Antibody were used for cell staining. Results are shown in FIGS. 7A-7F and FIGS. 8A-8F. Specifically, expression of mouse TNFR2 was detected in wild-type mouse spleen cells (FIG. 7C and FIG. 8C), but expression of human or humanized TNFR2 was not detected in wild-type mouse spleen cells (FIG. 7E and FIG. 8E). In addition, expressions of both mouse TNFR2 (FIG. 7D and FIG. 8D) and humanized TNFR2 (FIG. 7F and FIG. 8F) were detected in TNFR2 gene humanized heterozygous mice. Further analysis found that the cells expressing TNFR2 protein in the spleens of humanized and wild-type mice were consistent with comparable expression levels, indicating that TNFR2 can be expressed normally in TNFR2 gene humanized mice.

In a separate experiment, two 4-6 week old wild-type C57BL/6 mice and two 4-6 week old TNFR2 gene humanized homozygous mice were selected. One wild-type mouse and one TNFR2 homozygous mouse were stimulated by intraperitoneal injection of an anti-mCD3 antibody (mCD3). Then, all mice were tested for expression of TNFR2 protein on various immune cells in the mouse spleen according to the same methods below. First, anti-mTcRβPerCP/Cy5.5, anti-mCD19 FITC, anti-mFoxp3-APC, and anti-mCD4-BV421 were used to label cells. Then, the cells were stained by anti-mTNFR2 PE or anti-hTNFR2 PE, followed by flow cytometry analysis.

As described herein, T cells are defined as intact, single, live, CD19−, and TCR+; CD4+ T cells are defined as intact, single, live, CD19−, TCR+, and CD4+; Treg cells are defined as intact, single, live, CD19−, TCR+, CD4+, and Foxp3+; B cells are defined as intact, single, live, CD19+, and TCR−.

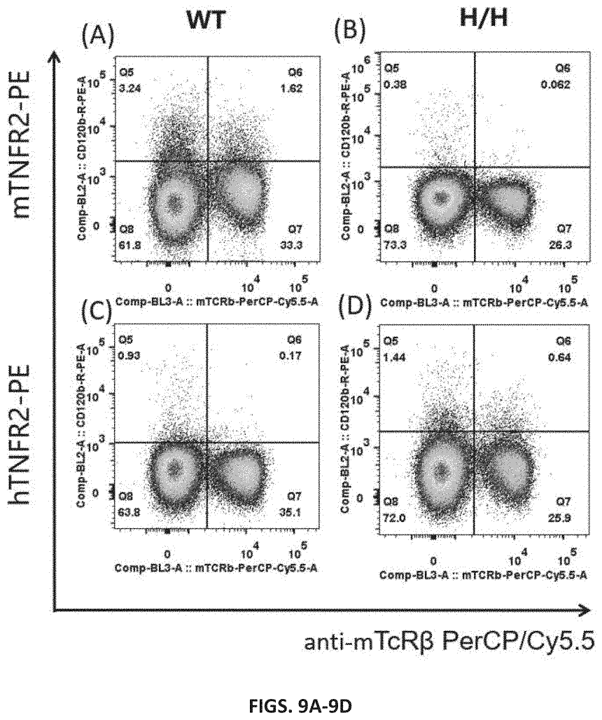

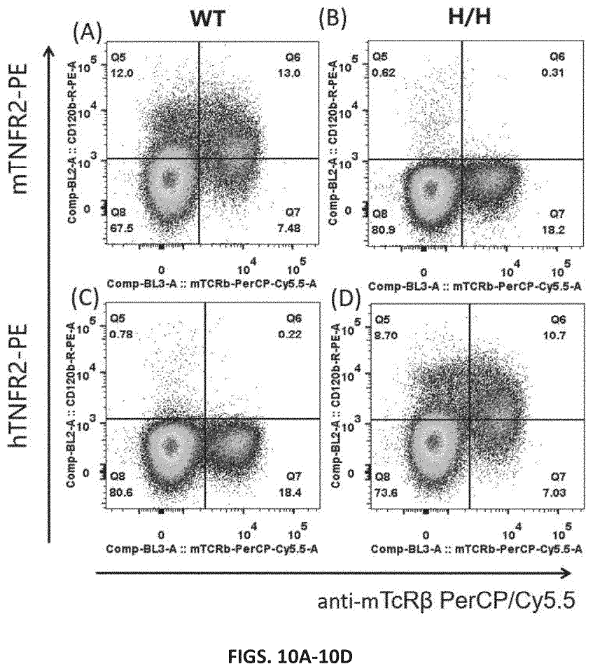

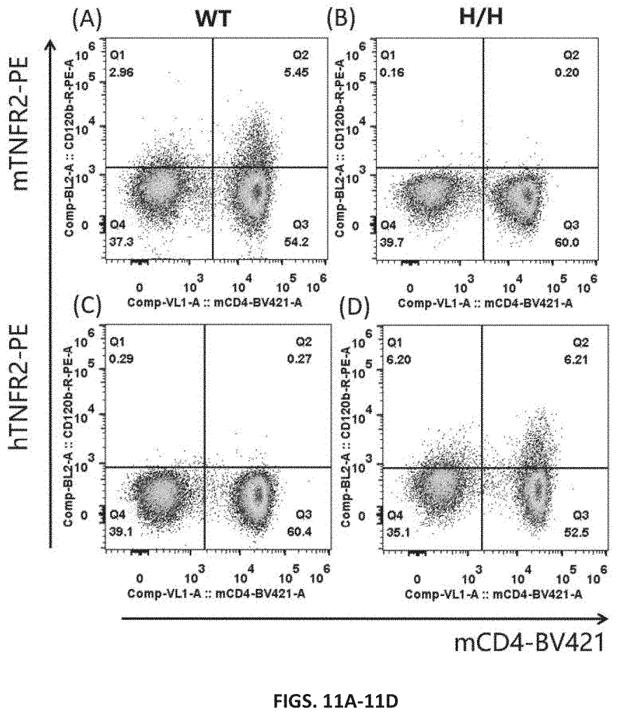

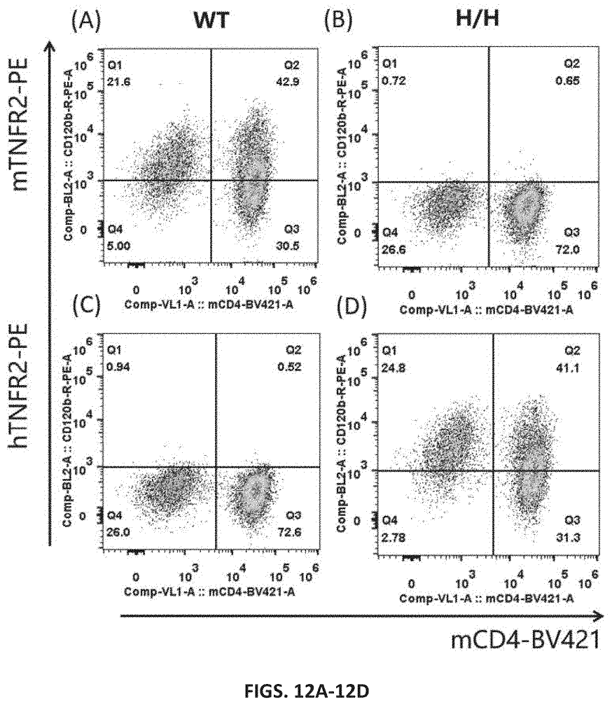

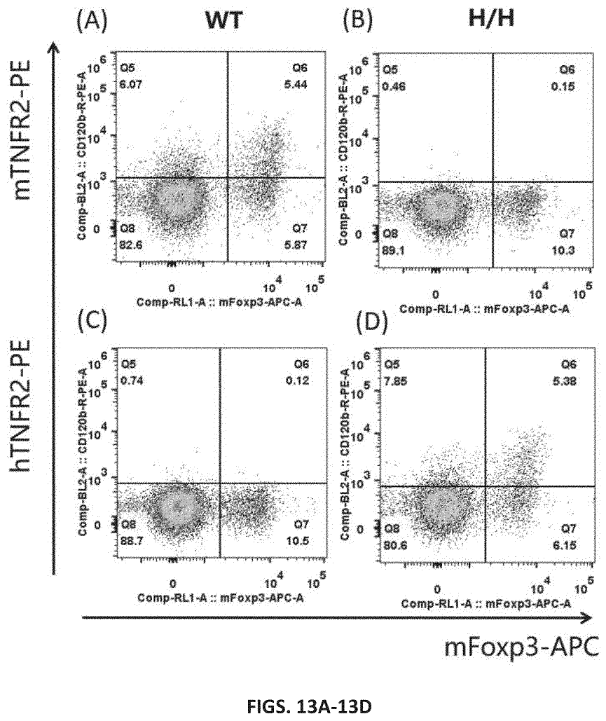

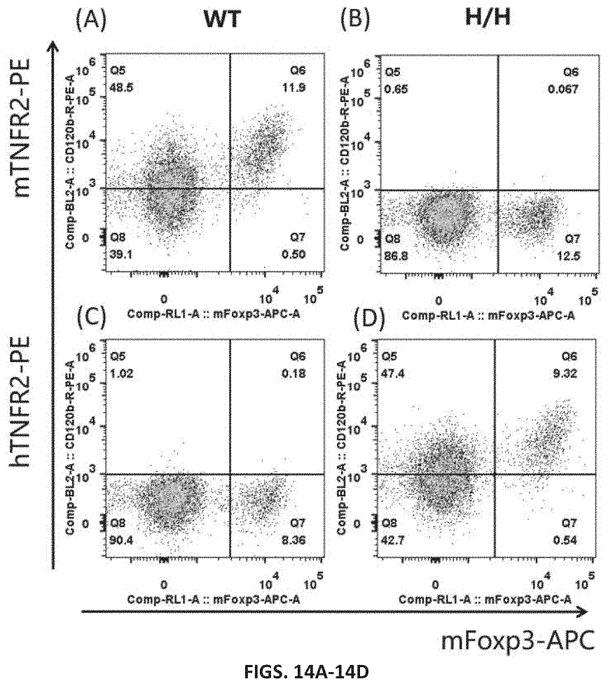

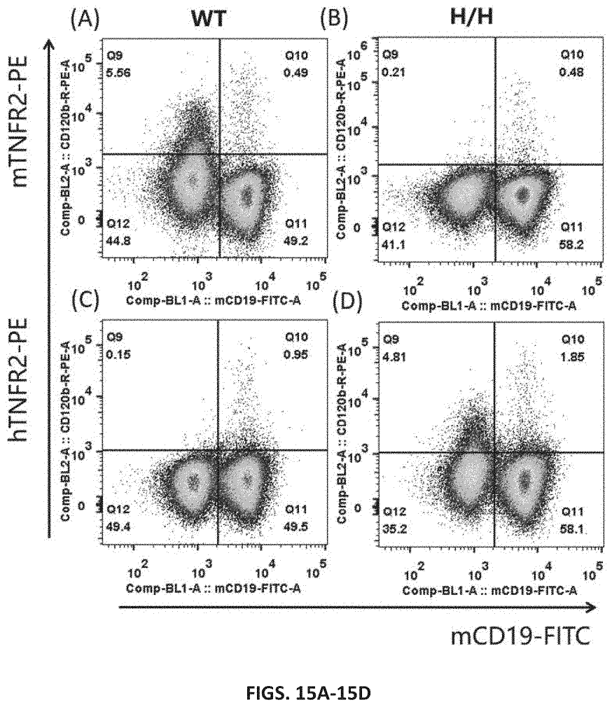

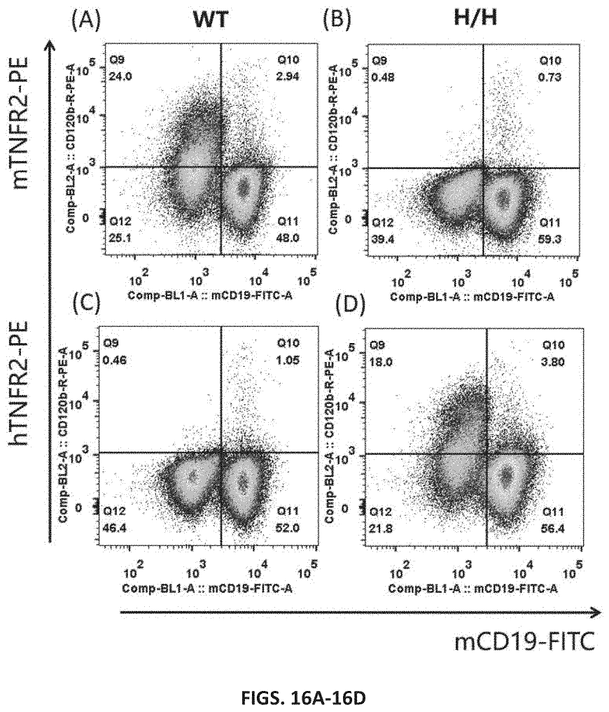

The results of flow cytometry showed that the expression of mouse TNFR2 was detected in T cells, CD4+ T cells and B cells in the spleen cells of wild-type mice (FIGS. 9A, 10A, 11A, 12A, 13A, 14A, 15A, and 16A). However, expression of human or humanized TNFR2 was not detected in the same cells of wild-type mice (FIGS. 9C, 10C, 11C, 12C, 13C, 14C, 15C, and 16C). In addition, expression of humanized TNFR2 was detected in homozygous cells of TNFR2 gene humanized mice (FIGS. 9D, 10D, 11D, 12D, 13D, 14D, 15D, and 16D). However, expression of mouse TNFR2 was not detected in the same cells of the TNFR2 gene humanized mice (FIGS. 9B, 10B, 11B, 12B, 13B, 14B, 15B, and 16B). After anti-mCD3 antibody stimulation, the expression of TNFR2 on the surface of immune cells in both wild-type and TNFR2 gene humanized mice increased to varying degrees. Among them, T cells and CD4+ T cells increased significantly (FIGS. 10A-10D, 12A-12D, and 14A-14D).

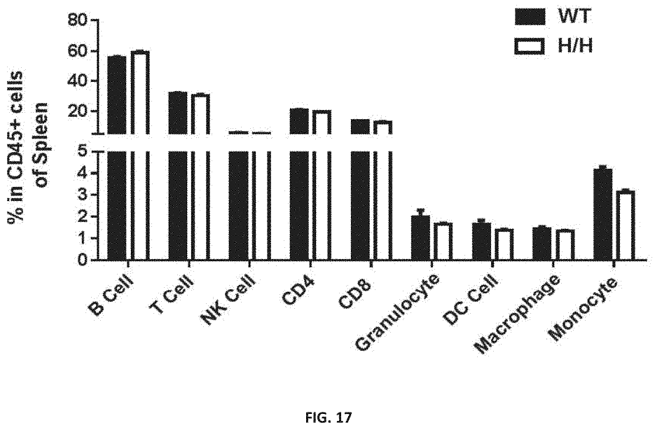

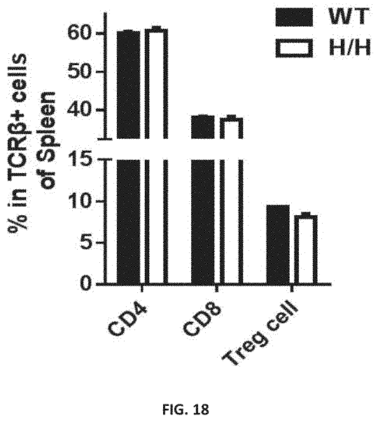

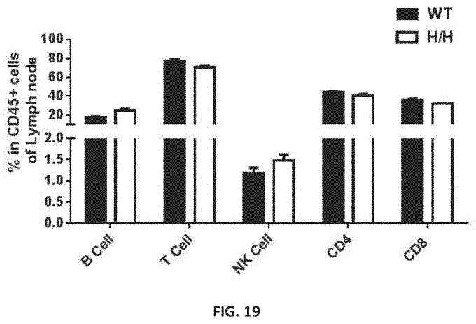

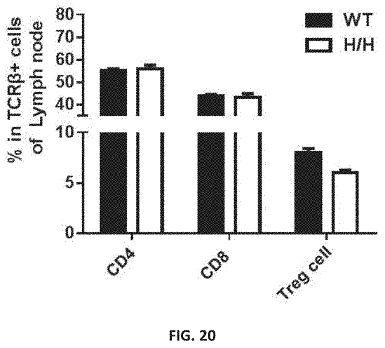

Furthermore, the immune cell grouping in the spleen and lymph nodes of TNFR2 gene humanized mice was also tested. No significant difference was found compared with the wild-type (FIGS. 17-20).

In another experiment, RNA extraction was performed from wild-type and TNFR2 gene humanized mice, respectively. Relative quantification of gene expression by real-time PCR was used to determine whether the TNFR2 gene humanization had an effect on the expression of the TNFR2 gene. Total RNA was extracted from the cells by the RNAprep pure Kit and then reverse transcribed to cDNA. Afterwards, PowerUp™ SYBR™ Green Master Mix kit and primers were used to amplify the target gene by PCR. The Applied BiosystemsQuantStudio™ 5 real-time PCR system was used for PCR amplification, with primer sequences as follows.

| | F: |
|---|---|
| (SEQ ID NO: 30) | |
| | 5′-GCCTACAAAGAGATGCCAAGGTG-3′; |
| | R: |
| (SEQ ID NO: 31) | |
| | 5′-TCTGAAATCCTGGAGCTGGCAC-3′. |

After the reaction, the amplification curve and dissolution curve of real-time PCR were obtained, and ΔΔCT was utilized for relative quantification of gene expression. The calculation formula is set forth below:

Relative gene expression=2^{-ΔΔCt}

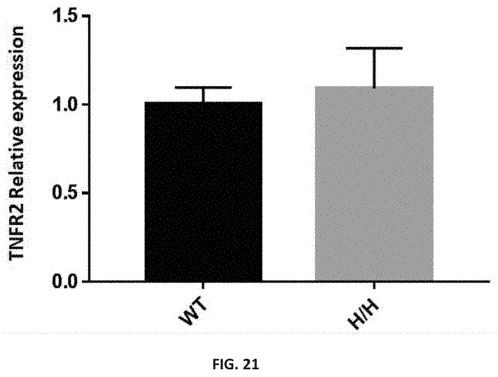

As shown in FIG. 21, compared with the wild-type TNFR2 expression level, the expression level of TNFR2 gene did not change significantly after the humanization of TNFR2 gene.

### Example 2: Generation and Use of Double- or Multi-Gene Humanized Mice

The humanized TNFR2 mouse prepared by the method described herein can also be used to prepare a double- or multi-gene humanized mouse model. For example, embryonic stem cells or fertilized eggs used in the microinjection and embryo transfer process are selected from other genetically modified mice. Alternatively, the embryonic stem cells or fertilized eggs of TNFR2 gene humanized mice are selected for gene editing, to obtain a double-gene or multi-gene humanized mouse model comprising humanized TNFR2 gene and other genetic modifications. In addition, it is also possible to mate the homozygous or heterozygous TNFR2 transgenic mice obtained by the method described herein with other genetically modified homozygous or heterozygous mice, and the offspring can be screened. According to Mendel's law, it is possible to generate double-gene or multi-gene modified heterozygous mice comprising humanized TNFR2 gene and other genetic modifications. Then the heterozygous mice can be mated with each other to obtain homozygous double-gene or multi-gene humanized mice.

### Example 3: In Vitro Test of Humanized Mouse Cells

Anti-human TNFR2 antibodies can be used to confirm whether TNFR2 signaling pathway function in the humanized mice is normal. As verified by preliminary experiments, three antibodies Ab1, Ab2, and Ab3 are all anti-human TNFR2 antibodies.

Spleen cells from wild-type C57BL/6 mice (WT) and TNFR2 gene humanized homozygous mice (H/H) were divided into two groups and stimulated with mCD3E, mCD28 and/or mTNFα. Cells from each source were divided into 5 fractions, 3 of which were randomly selected and added with 200 nM of the three antibodies Ab1, Ab2, Ab3, respectively. The other 2 fractions were randomly added with anti-mouse PD-1 antibody (mPD-1 Ab) (as a positive control), and added with the same amount of IgG1 (as an isotype control), respectively. In particular, mPD-1 Ab was verified to bind to mouse PD-1 and can inhibit tumor growth.

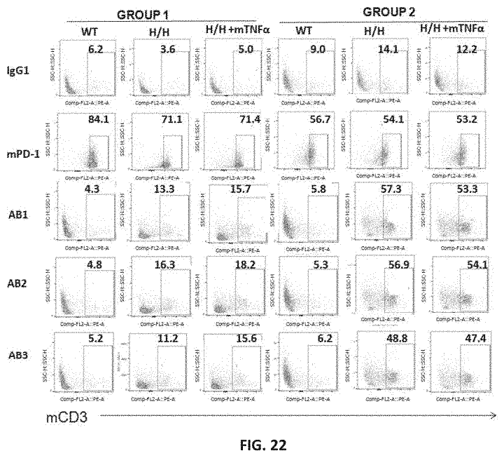

Flow cytometry was used to detect the binding of the added antibodies to TNFR2 or PD-1 on the cells. As shown in FIG. 22, the test results showed that the three anti-human TNFR2 antibodies can bind to the TNFR2 gene humanized homozygous mouse cells, but not the wild-type C57BL/6 mouse cells. Under low mCD3E/mCD28 stimulation conditions, the addition of mTNFα (ligand of TNFR2) significantly enhanced the binding of antibodies to TNFR2 on the surface of CD3+ T cells. In addition, the addition of mTNFα activated humanized TNFR2 cells and increased the expression of TNFR2 on the cell surface.

TABLE 5
Binding test protocol

| | Cell Source | Stimulants (concentration) |
|---|---|---|
| Group 1 | WT | mCD3E (0.2 μg/mL), mCD28 (1 μg/mL) |
| | H/H | mCD3E (0.2 μg/mL), mCD28 (1 μg/mL) |
| | H/H | mCD3E (0.2 μg/mL), mCD28 (1 μg/mL), mTNFcα (25 ng/mL) |
| Group 2 | WT | mCD3E (1 μg/mL), mCD28 (1 μg/mL) |
| | H/H | mCD3E (1 μg/mL), mCD28 (1 μg/mL) |
| | H/H | mCD3E (1 μg/mL), mCD28 (1 μg/mL), mTNFα (25 ng/mL) |

Furthermore, the NFκB signaling pathway in different immune cells after the addition of antibodies was also tested in vitro. It was found that after stimulation with different anti-human TNFR2 antibodies, cells isolated from the humanized mice showed different levels of NFκB signals, and T cells showed different levels of activation. The overall results were consistent with the results using human PBMC (data not shown).

This study proved that the TNFR2 signaling pathway in the humanized mice functioned normally, and the expressed humanized TNFR2 protein can bind to anti-human TNFR2 antibodies and mouse TNFα. The results also showed that the mice prepared by the method described herein can be used for the screening of anti-human TNFR2 blocking antibodies, and evaluation of drug efficacy.

### Example 4: Pharmacological Validation of TNFR2 Gene Humanized Mouse Model

In one experiment, the TNFR2 gene humanized homozygous mice (9-10 week old) were subcutaneously injected with mouse colon cancer cell MC38 (5×10⁵ cells/100 μl PBS). After the tumor volume reached about 50-100 mm³, the mice were randomly divided into 5 groups (8 mice in each group) according to the tumor volume. Four groups were randomly selected for the three anti-human TNFR2 antibody (Ab1, Ab2, and Ab3) treatment and one anti-mouse PD-1 antibody (mPD-1 Ab) treatment. The control group mice were injected with an equal volume of isotype control IgG1 (simplified as Iso-type). Specific grouping and method of administration are shown in Table 6 below. The antibodies were intraperitoneally (i.p.) injected at 10 mg/kg. The tumor volume was measured twice a week and body weight of the mice was recorded as well. Euthanasia was performed when tumor volume of a mouse reached 3000 mm³.

TABLE 6

| Group | Antibody | Administration Route and Frequency |
|---|---|---|
| G1 | IgG1 (Iso-type) | i.p., every 3 days, 7 times in total |
| G2 | mPD-1 Ab | i.p., every 3 days, 4 times in total |
| G3 | Ab1 | i.p., every 3 days, 7 times in total |
| G4 | Ab2 | i.p., every 3 days, 7 times in total |
| G5 | Ab3 | i.p., every 3 days, 7 times in total |

Table 5 shows results for this experiment, including the tumor volumes at Day 0, Day 14, and Day 21 (the last day of the experiment) after the grouping; the survival rate of the mice; the Tumor Growth Inhibition value (TGI_{TV} %); and the statistical differences (P value) in mouse body weights and tumor volume between the treatment and control groups.

TABLE 7

| | | | | | | | P value | |
|---|---|---|---|---|---|---|---|---|
| | | Tumor volume(mm³) | | | | | Body | Tumor |
| | | Day 0 | Day 14 | Day 21 | Survival | TGI_{TV} % | weight | Volume |
| Control | G1 | 89 ± 2 | 941 ± 93 | 1832 ± 134 | 8/8 | N/A | N/A | N/A |
| Treatment | G2 (mPD-1 | 89 ± 2 | 394 ± 59 | 794 ± 109 | 8/8 | 59.6 | 0.93 | 3.23 × 10⁻⁵ |
| groups | Ab) | | | | | | | |
| | G3 (Ab1) | 89 ± 2 | 513 ± 81 | 981 ± 154 | 8/8 | 48.9 | 0.88 | 0.001 |
| | G4 (Ab2) | 89 ± 2 | 650 ± 130 | 1194 ± 251 | 8/8 | 36.6 | 0.94 | 0.041 |
| | G5 (Ab3) | 89 ± 3 | 424 ± 78 | 665 ± 146 | 8/8 | 67.0 | 0.59 | 3.93 × 10⁻⁵ |

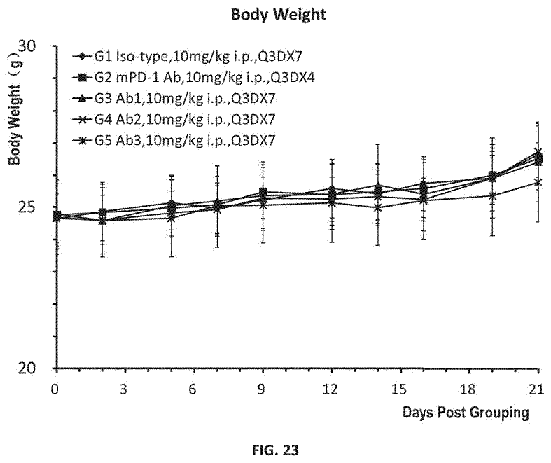

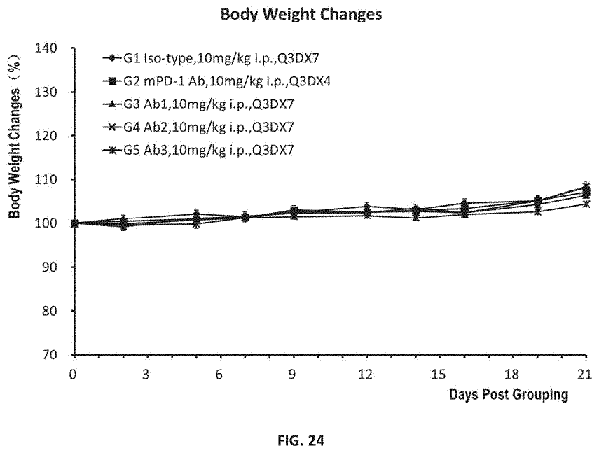

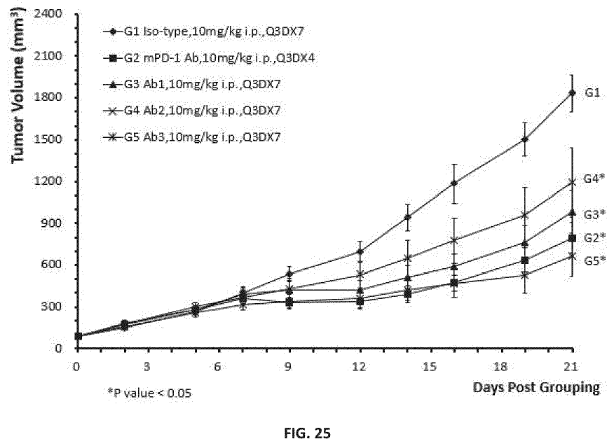

Overall, the mice in each group were grossly healthy. At the end of the experiment (day 21), the body weight of each group increased and there was no significant difference between the groups (FIGS. 23 and 24). However, with respect to the tumor volume (FIG. 25), the tumors of the control group mice continued to grow during the experimental period, while the tumor volume growth of all the treatment group mice showed different degrees of inhibition and/or shrinkage compared with the control group. Thus, this indicates that the three anti-human TNFR2 antibodies and anti-mouse PD-1 antibody were well tolerated, and the antibodies inhibited the tumor growth in mice.

Specifically, with respect to the measured tumor volume (FIG. 25 and Table 7), the average tumor volume in the control group (G1) was 1832±134 mm³. The average tumor volumes in the treatment groups were 794±109 mm³ (G2), 981±154 mm³ (G3), 1194±251 mm³ (G4), and 665±146 mm³ (G5), respectively. The tumor volume in each treatment group was significantly smaller (P value≤0.05) than that in the control group (G1), showing that the three anti-human TNFR2 antibodies and anti-mouse PD-1 antibody had different tumor growth inhibitory effects. Specifically, at the same dosage, the anti-human TNFR2 antibody Ab3 exhibited the best and most obvious tumor growth inhibitory effect (67% for TGI_{TV} %), which was slightly more effective than the anti-mouse PD-1 antibody mPD-1 Ab (TGI_{TV} % was 59.6%). In addition, Ab3 exhibited a stronger tumor inhibitory effect than Ab1 and Ab2, in treating and inhibiting tumor growth in TNFR2 gene humanized mice. The results proved that the anti-human TNFR2 antibodies exhibited different tumor growth inhibition effects in TNFR2 gene humanized mice, and none of the antibodies showed obvious toxic effects to the animals.

The above experiments demonstrated that the humanized TNFR2 animal model can be used as a living model for in vivo drug efficacy research; screening, evaluation, and treatment experiments of TNFR2-associated modulators; evaluation of effectiveness and therapeutic effects of antibodies targeting human TNFR2 signaling pathway in animals.

### Example 5: Methods Based on CRISPR

The non-human mammals described herein can also be prepared through other gene editing systems and approaches, including but not limited to: clustered regularly interspaced short palindromic repeats (CRISPR), zinc finger nuclease (ZFN), transcriptional activator-like effector factor nuclease (TALEN), homing endonuclease (megakable base ribozyme), or some other techniques. The example herein uses CRISPR as an example to explain how to prepare and obtain TNFR2 gene humanized mice by some other methods.

The present disclosure is related to replacing all or part of exons 2-6 of mouse TNFR2 gene with human TNFR2 gene fragments in situ. Thus, sgRNAs with different targeting sites are designed and their cleavage efficiencies are verified using a UCA kit. The sgRNAs with the highest activities are selected for downstream experiments. Further, a recombinant vector containing a 5′ homologous arm, a 3′ homologous arm, and a humanized gene fragment is designed. Construction of the vector can be carried out by routine methods, e.g., restriction enzyme digestion and ligation. Next, the verified recombination vector, and in vitro transcription products of the selected sgRNAs are injected into the cytoplasm or nucleus of mouse fertilized eggs (C57BL/6 background) with a microinjection instrument. The embryo microinjection can be carried out according to the method described, e.g., in A. Nagy, et al., “Manipulating the Mouse Embryo: A Laboratory Manual (Third Edition),” Cold Spring Harbor Laboratory Press, 2003. The injected fertilized eggs are then transferred to a culture medium for a short time culture, and then is transplanted into the oviduct of the recipient mouse to produce the genetically modified humanized mice (F0 generation). The F0 generation chimeric mice with correct gene recombination are then selected by extracting the mouse tail genome and detecting by PCR for subsequent breeding and identification. F1 generation mice are obtained by mating F0 positive mice with wild-type mice. By extracting the mouse tail genome and PCR detection again, positive F1 generation heterozygous mice are selected with stable genetic recombination. Next, the F1 generation heterozygous mice are mated to each other to obtain genetically recombinant positive F2 generation homozygous mice.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human animal whose genome comprises at least one chromosome comprising a sequence encoding a human or chimeric tumor necrosis factor receptor 2 (TNFR2).

2. The animal of claim 1, wherein the sequence encoding the human or chimeric TNFR2 is operably linked to an endogenous regulatory element at the endogenous TNFR2 gene locus in the at least one chromosome.

3. The animal of claim 1, wherein the sequence encoding a human or chimeric TNFR2 comprises a sequence encoding an amino acid sequence that is at least 70%, 75%, 80%, 85%, 90%, 95%, 99%, or 100% identical to human TNFR2 (NP_001057.1 (SEQ ID NO: 4)).

4. The animal of claim 1, wherein the sequence encoding a human or chimeric TNFR2 comprises a sequence encoding an amino acid sequence that is at least 70%, 75%, 80%, 85%, 90%, 95%, 99%, or 100% identical to SEQ ID NO: 9.

5. The animal of claim 1, wherein the sequence encoding a human or chimeric TNFR2 comprises a sequence that is at least 70%, 75%, 80%, 85%, 90%, 95%, 99%, or 100% identical to amino acids 33-259 of SEQ ID NO: 4.

6. The animal of any one of claims 1-5, wherein the animal is a mammal, e.g., a monkey, a rodent, or a mouse.

7. The animal of any one of claims 1-5, wherein the animal is a mouse.

8. The animal of any one of claims 1-7, wherein the animal does not express endogenous TNFR2.

9. The animal of claim 1, wherein the animal has one or more cells expressing human or chimeric TNFR2.

10. The animal of claim 1, wherein the animal has one or more cells expressing human or chimeric TNFR2, and a human TNFα can bind to the expressed human or chimeric TNFR2.

11. The animal of claim 1, wherein the animal has one or more cells expressing human or chimeric TNFR2, and an endogenous TNFα can bind to the expressed human or chimeric TNFR2.

12. A genetically-modified, non-human animal, wherein the genome of the animal comprises a replacement of a sequence encoding a region of endogenous TNFR2 with a sequence encoding a corresponding region of human TNFR2 at an endogenous TNFR2 gene locus.

13. The animal of claim 12, wherein the sequence encoding the corresponding region of human TNFR2 is operably linked to an endogenous regulatory element at the endogenous TNFR2 locus, and one or more cells of the animal expresses a chimeric TNFR2.

14. The animal of claim 12, wherein the animal does not express endogenous TNFR2.

15. The animal of claim 12, wherein the replaced locus is the extracellular region of TNFR2.

16. The animal of claim 12, wherein the animal has one or more cells expressing a chimeric TNFR2 having an extracellular region, a transmembrane region, and a cytoplasmic region, wherein the extracellular region comprises a sequence that is at least 50%, 60%, 70%, 80%, 90%, 95%, or 99% identical to the extracellular region of human TNFR2.

17. The animal of claim 16, wherein the extracellular region of the chimeric TNFR2 has a sequence that has at least 10, 20, 30, 40, 50, 60, 70, 80, 90, or 100 contiguous amino acids that are identical to a contiguous sequence present in the extracellular region of human TNFR2.

18. The animal of claim 12, wherein the animal is a mouse, and the sequence encoding the region of endogenous TNFR2 is exon 2, exon 3, exon 4, exon 5, and/or exon 6 of the endogenous mouse TNFR2 gene.

19. The animal of claim 12, wherein the animal is heterozygous with respect to the replacement at the endogenous TNFR2 gene locus.

20. The animal of claim 12, wherein the animal is homozygous with respect to the replacement at the endogenous TNFR2 gene locus.

21. A method for making a genetically-modified, non-human animal, comprising:
replacing in at least one cell of the animal, at an endogenous TNFR2 gene locus, a sequence encoding a region of an endogenous TNFR2 with a sequence encoding a corresponding region of human TNFR2.

22. The method of claim 21, wherein the sequence encoding the corresponding region of human TNFR2 comprises exon 1, exon 2, exon 3, exon 4, exon 5, exon 6, exon 7, exon 8, exon 9, and/or exon 10 of a human TNFR2 gene.

23. The method of claim 21, wherein the sequence encoding the corresponding region of TNFR2 comprises exon 2, exon 3, exon 4, exon 5, and/or exon 6, or part thereof, of a human TNFR2 gene.

24. The method of claim 21, wherein the sequence encoding the corresponding region of human TNFR2 encodes amino acids 33-259 of SEQ ID NO: 4.

25. The method of claim 21, wherein the region is located within the extracellular region of TNFR2.

26. The method of claim 21, wherein the animal is a mouse, and the endogenous TNFR2 locus is exon 2, exon 3, exon 4, exon 5, exon 6, exon 7, exon 8, exon 9, and/or exon 10 of the mouse TNFR2 gene.

27. The method of claim 21, wherein the animal is a mouse, and the endogenous TNFR2 locus is exon 2, exon 3, exon 4, exon 5, and/or exon 6 of the mouse TNFR2 gene.

28. A non-human animal comprising at least one cell comprising a nucleotide sequence encoding a chimeric TNFR2 polypeptide, wherein the chimeric TNFR2 polypeptide comprises at least 50 contiguous amino acid residues that are identical to the corresponding contiguous amino acid sequence of a human TNFR2, wherein the animal expresses the chimeric TNFR2.

29. The animal of claim 28, wherein the chimeric TNFR2 polypeptide has at least 50 contiguous amino acid residues that are identical to the corresponding contiguous amino acid sequence of a human TNFR2 extracellular region.

30. The animal of claim 28, wherein the chimeric TNFR2 polypeptide comprises a sequence that is at least 90%, 95%, or 99% identical to amino acids 33-259 of SEQ ID NO: 4.

31. The animal of claim 28, wherein the nucleotide sequence is operably linked to an endogenous TNFR2 regulatory element of the animal.

32. The animal of claim 28, wherein the chimeric TNFR2 polypeptide comprises an endogenous TNFR2 transmembrane region and/or an endogenous TNFR2 cytoplasmic region.

33. The animal of claim 28, wherein the nucleotide sequence is integrated to an endogenous TNFR2 gene locus of the animal.

34. The animal of claim 28, wherein the chimeric TNFR2 has at least one mouse TNFR2 activity and/or at least one human TNFR2 activity.

35. A method of making a genetically-modified mouse cell that expresses a chimeric TNFR2, the method comprising:
replacing at an endogenous mouse TNFR2 gene locus, a nucleotide sequence encoding a region of mouse TNFR2 with a nucleotide sequence encoding a corresponding region of human TNFR2, thereby generating a genetically-modified mouse cell that includes a nucleotide sequence that encodes the chimeric TNFR2, wherein the mouse cell expresses the chimeric TNFR2.

36. The method of claim 35, wherein the chimeric TNFR2 comprises:
an extracellular region of human TNFR2 comprising a mouse signal peptide sequence; and
a transmembrane and/or a cytoplasmic region of mouse TNFR2.

37. The method of claim 36, wherein the nucleotide sequence encoding the chimeric TNFR2 is operably linked to an endogenous TNFR2 regulatory region, e.g., promoter.

38. The animal of any one of claims 1-20 and 28-34, wherein the animal further comprises a sequence encoding an additional human or chimeric protein.

39. The animal of claim 38, wherein the additional human or chimeric protein is tumor necrosis factor alpha (TNFα), programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (OX40).

40. The method of any one of claims 21-27 and 35-37, wherein the animal or mouse further comprises a sequence encoding an additional human or chimeric protein.

41. The method of claim 40, wherein the additional human or chimeric protein is TNFα, PD-1, CTLA-4, LAG-3, BTLA, PD-L1, CD27, CD28, CD47, CD137, CD154, TIGIT, TIM-3, GITR, SIRPα or OX40.

42. A method of determining effectiveness of an anti-TNFR2 antibody for the treatment of cancer, comprising:
a) administering the anti-TNFR2 antibody to the animal of any one of claims 1-20 and 28-34, wherein the animal has a tumor; and
b) determining the inhibitory effects of the anti-TNFR2 antibody to the tumor.

43. The method of claim 42, wherein the tumor comprises one or more cells that express TNFR2.

44. The method of claim 42, wherein the tumor comprises one or more cancer cells that are injected into the animal.

45. The method of claim 42, wherein determining the inhibitory effects of the anti-TNFR2 antibody to the tumor involves measuring the tumor volume in the animal.

46. The method of claim 42, wherein the tumor cells are breast cancer cells, colon cancer cells, cervical cancer cells, fibrosarcoma, liver cancer cells, lung cancer cells, melanoma cells, ovarian cancer cells, renal cancer cells, skin cancer cells, plasmacytoma, lymphoma, or leukemia.

47. A method of determining effectiveness of an anti-TNFR2 antibody and an additional therapeutic agent for the treatment of a tumor, comprising
a) administering the anti-TNFR2 antibody and the additional therapeutic agent to the animal of any one of claims 1-20 and 28-34, wherein the animal has a tumor; and
b) determining the inhibitory effects on the tumor.

48. The method of claim 47, wherein the animal further comprises a sequence encoding a human or chimeric programmed cell death protein 1 (PD-1).

49. The method of claim 47, wherein the animal further comprises a sequence encoding a human or chimeric programmed death-ligand 1 (PD-L1).

50. The method of claim 47, wherein the additional therapeutic agent is an anti-PD-1 antibody or an anti-PD-L1 antibody.

51. The method of claim 47, wherein the tumor comprises one or more tumor cells that express TNFR2, PD-1 or PD-L1.

52. The method of claim 47, wherein the tumor is caused by injection of one or more cancer cells into the animal.

53. The method of claim 47, wherein determining the inhibitory effects of the treatment involves measuring the tumor volume in the animal.

54. The method of claim 47, wherein the animal has breast cancer, colon cancer, cervical cancer, fibrosarcoma, liver cancer, lung cancer, non-small cell lung cancer (NSCLC), melanoma, ovarian cancer, renal cancer, skin cancer, plasmacytoma, lymphoma, and/or leukemia.

55. A method of determining effectiveness of an anti-TNFR2 antibody for treating an autoimmune disorder, comprising:
a) administering the anti-TNFR2 antibody to the animal of any one of claims 1-20 and 28-34, wherein the animal has the autoimmune disorder; and
b) determining effects of the anti-TNFR2 antibody for treating the auto-immune disease.

56. The method of claim 55, wherein the autoimmune disorder is rheumatoid arthritis, Crohn's disease, systemic lupus erythematosus, ankylosing spondylitis, inflammatory bowel diseases (IBD), ulcerative colitis, and/or scleroderma.

57. A method of determining effectiveness of an anti-TNFR2 antibody for treating an immune disorder, comprising:
a) administering the anti-TNFR2 antibody to the animal of any one of claims 1-20 and 28-34, wherein the animal has the immune disorder; and
b) determining effects of the anti-TNFR2 antibody for treating the immune disease.

58. The method of claim 57, wherein the immune disorder is allergy, asthma, and/or atopic dermatitis.

59. A protein comprising an amino acid sequence, wherein the amino acid sequence is one of the following:
(a) an amino acid sequence set forth in SEQ ID NO: 9;
(b) an amino acid sequence that is at least 90% identical to SEQ ID NO: 9;
(c) an amino acid sequence that is at least 91%, 92%, 93%, 94%, 95%, 96%, 97%, 98%, or 99% identical to SEQ ID NO: 9;
(d) an amino acid sequence that is different from the amino acid sequence set forth in SEQ ID NO: 9 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 9.

60. A nucleic acid comprising a nucleotide sequence, wherein the nucleotide sequence is one of the following:
(a) a sequence that encodes the protein of claim 59;
(b) SEQ ID NO: 7, SEQ ID NO: 8, SEQ ID NO: 10, or SEQ ID NO: 11;
(c) a sequence that is at least 90% identical to SEQ ID NO: 7, 8, 10, or 11;
(d) a sequence that is at least 91%, 92%, 93%, 94%, 95%, 96%, 97%, 98%, or 99% identical to SEQ ID NO: 7, 8, 10, or 11; and
(e) a sequence that is at least 91%, 92%, 93%, 94%, 95%, 96%, 97%, 98%, or 99% identical to SEQ ID NO: 7, 8, 10, or 11.

61. A cell comprising the protein of claim 59 and/or the nucleic acid of claim 60.

62. An animal comprising the protein of claim 59 and/or the nucleic acid of claim 60.


SEQUENCE LISTING (32 sequences)

SEQ ID NO: 1 - DNA, 4913 bp, Mus musculus
agtcaccagctagagcgcagctgaggcactagagctccaggcacaagggcgggagccaccgctgcccctatggcgcccgccgccctctgggtcgcgctggtcttcgaactgcagctgtgggccaccgggcacacagtgcccgcccaggttgtcttgacaccctacaaaccggaacctgggtacgagtgccagatctcacaggaatactatgacaggaaggctcagatgtgctgtgctaagtgtcctcctggccaatatgtgaaacatttctgcaacaagacctcggacaccgtgtgtgcggactgtgaggcaagcatgtatacccaggtctggaaccagtttcgtacatgtttgagctgcagttcttcctgtaccactgaccaggtggagatccgcgcctgcactaaacagcagaaccgagtgtgtgcttgcgaagctggcaggtactgcgccttgaaaacccattctggcagctgtcgacagtgcatgaggctgagcaagtgcggccctggcttcggagtggccagttcaagagccccaaatggaaatgtgctatgcaaggcctgtgccccagggacgttctctgacaccacatcatccactgatgtgtgcaggccccaccgcatctgtagcatcctggctattcccggaaatgcaagcacagatgcagtctgtgcgcccgagtccccaactctaagtgccatcccaaggacactctacgtatctcagccagagcccacaagatcccaacccctggatcaagagccagggcccagccaaactccaagcatccttacatcgttgggttcaacccccattattgaacaaagtaccaagggtggcatctctcttccaattggtctgattgttggagtgacatcactgggtctgctgatgttaggactggtgaactgcatcatcctggtgcagaggaaaaagaagccctcctgcctacaaagagatgccaaggtgcctcatgtgcctgatgagaaatcccaggatgcagtaggccttgagcagcagcacctgttgaccacagcacccagttccagcagcagctccctagagagctcagccagcgctggggaccgaagggcgccccctgggggccatccccaagcaagagtcatggcggaggcccaagggtttcaggaggcccgtgccagctccaggatttcagattcttcccacggaagccacgggacccacgtcaacgtcacctgcatcgtgaacgtctgtagcagctctgaccacagttctcagtgctcttcccaagccagcgccacagtgggagacccagatgccaagccctcagcgtccccaaaggatgagcaggtccccttctctcaggaggagtgtccgtctcagtccccgtgtgagactacagagacactgcagagccatgagaagcccttgccccttggtgtgccggatatgggcatgaagcccagccaagctggctggtttgatcagattgcagtcaaagtggcctgacccctgacaggggtaacaccctgcaaagggacccccgagaccctgaacccatggaacttcatgacttttgctggatccatttcccttagtggcttccagagccccagttgcaggtcaagtgagggctgagacagctagagtggtcaaaaactgccatggtgttttatgggggcagtcccaggaagttgttgctcttccatgacccctctggatctcctgggctcttgcctgattcttgcttctgagaggccccagtattttttccttctaaggagctaacatcctcttccatgaatagcacagctcttcagcctgaatgctgacactgcagggcggttccagcaagtaggagcaagtggtggcctggtagggcacagaggcccttcaggttagtgctaaactcttaggaagtaccctctccaagcccaccgaaattcttttgatgcaagaatcagaggccccatcaggcagagttgctctgttataggatggtagggctgtaactcagtggtccagtgtgcttttagcatgccctgggtttgatcctcagcaacacatgcaaaacgtaagtagacagcagacagcagacagcacagccagccccctgtgtggtttgcagcctctgcctttgacttttactctggtgggcacacagagggctggagctcctcctcctgaccttctaatgagcccttccaaggccacgccttccttcagggaatctcagggactgtagagttcccaggcccctgcagccacctgtctcttcctacctcagcctggagcactccctctaactccccaacggcttggtactgtacttgctgtgaccccaagtgcatgtccgggttaggcactgtgagttggaacagctgatgacatcggttgaaaggcccacccggaaacagctgaagccagctcttttgccaaaggattcatgccggttttctaatcaacctgctcccctagcatgcctggaaggaaagggttcaggagactcctcaagaagcaagttcagtctcaggtgcttggatgccatgctcaccgattccactggatatgaacttggcagaggagcctagttgttgccatggagacttaaagagctcagcactctggaatcaagatactggacacttggggccgacttgttaaggctctgcagcatcagactgtagaggggaaggaacacgtctgccccctggtggcccgtcctgggatgacctcgggcctcctaggcaacaaaagaatgaattggaaaggactgttcctgggtgtggcctcagctcctgtgcttgtgtggatccctaaagggtgtgctaaggagcaattgcactgtgtgctggacagaattcctgcttataaatgctttttgttgttgttttgtacactgagccctggctgagccaccccaccccacctcccatcccacctttacagccactcttgcagagaacctggctgtctcccacttgtagcctgtggatgctgaggaaacacccagccaagtagactccaggcttgcccctatctcctgctctgagtctggcctcctcattgtgttgtgggaaggagacgggttctgtcatctcggaagcccacaccgtggatgtgaacaatggctgtactagcttagaccagcttagggctctgcaatcagaggagggggagcagggaacaatttgagtgctgacctataacacattcctaaaggatgggcagtccagaatctccctccttcagtgtgtgtgtgtgtgtgtgtgtgtgtgtgtgtgtgtgtgtgtccatgtttgcatgtatgtgtgtgccagtgtgtggaggccagaggttggctttgggtgtgtttgatcactctcagttactgaggcagggctctcatctgtacccagagcttgcacattttctagtctaacttgcttcagggatctctgtctgcctatggagtgctcaggttacaggcaggctgccatacctgcccgacatttacatgaatactagagatctgaattctggtcctcacacttgtatacctgcattttatccactaagacatctctccaagggctcccccttcctatttaataagttagttttgaactggcaagatggctcagtgggtaaggcagtttgcggacaaacctgatgacctgagttggatccctgaccataaggtagaagagacctgattcctgcaagttgtcctctgaccaccaccccatacatgcttctgcatatgtgcacacatcacattcttgcacacacactcacataccataaatgtaataaatttttttaaataaattgattttatcttttaatcattatttttgcttatctgtttgttcatacatggagttttgctgtatacccagtctggctgcatcctccaggcaccttgaacttggatattctcctgcctcagtttcgagagtgctgagttataggcatgtgcaaccgtgtcaggacccttggcgttacattatgttttcgaatcttatttttccatctaagactgacaaccaccttgccaaaggatgaccttgacttttgtatcctcttgcctctacttcccaagtattgggatgacaagtctgtccatcctatgatttgtgtggtgctggggcttgaacccaggggcctcgtgaatgctaggcaaacgcaatcagctgcccaaccccgaacttcacttttcacctgtgatttcttaacatggttcctgagcagacagcatgatcctgttgccttgaggagctctgctcgttgctttgacaatgctctatttactaaagaatacttatagagttcaagactccaaagcagcagttcccaacctgtgggtatgacctcttgggtatcccatatcagatatttacattaaacttataacagaagcaaaattacagttatggggtagcaacaaaataatttttttttgaggcagggtttatctgtgtagctcactttgtacagactaggctggccttgaacttaaagatctgcctgcctctgccttctgaatgctgggattaaatgtgtgtgccaacatgcccagtgcagtgaaataattttattgttgggtcaccaaaacatgagaaagagtattaaaaggtcatagcgttaggaaggttgagaaccactgccccaaaagatagtagaaataggtaatagcattttaaaaaaagattaatttctttttccgtgtgtgtgtgtgtgtgtgtgtgtgtgtgtgtgtgtgtgtgtgtgtgtgtgtgtgaagggtgctgaattccctaaatctgggattacagacaattgtgccaaatgggtattgggaaccaaagttgggttttctgaaagagcagcaagtgcccttaactgctgagccctctctctagctataataaacttttttttaaaaaaattaaaaaaatttagcactc

SEQ ID NO: 2 - protein, 474 aa, Mus musculus
MAPAALWVALVFELQLWATGHTVPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGISLPIGLIVGVTSLGLLMLGLVNCIILVQRKKKPSCLQRDAKVPHVPDEKSQDAVGLEQQHLLTTAPSSSSSSLESSASAGDRRAPPGGHPQARVMAEAQGFQEARASSRISDSSHGSHGTHVNVTCIVNVCSSSDHSSQCSSQASATVGDPDAKPSASPKDEQVPFSQEECPSQSPCETTETLQSHEKPLPLGVPDMGMKPSQAGWFDQIAVKVA

SEQ ID NO: 3 - DNA, 3682 bp, Homo sapiens
gcgagcgcagcggagcctggagagaaggcgctgggctgcgagggcgcgagggcgcgagggcagggggcaaccggaccccgcccgcacccatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaaccaggccggtgtgggctgtgtcgtagccaaggtgggctgagccctggcaggatgaccctgcgaaggggccctggtccttccaggcccccaccactaggactctgaggctctttctgggccaagttcctctagtgccctccacagccgcagcctccctctgacctgcaggccaagagcagaggcagcgagttgtggaaagcctctgctgccatggcgtgtccctctcggaaggctggctgggcatggacgttcggggcatgctggggcaagtccctgactctctgtgacctgccccgcccagctgcacctgccagcctggcttctggagcccttgggttttttgtttgtttgtttgtttgtttgtttgtttctccccctgggctctgccccagctctggcttccagaaaaccccagcatccttttctgcagaggggctttctggagaggagggatgctgcctgagtcacccatgaagacaggacagtgcttcagcctgaggctgagactgcgggatggtcctggggctctgtgcagggaggaggtggcagccctgtagggaacggggtccttcaagttagctcaggaggcttggaaagcatcacctcaggccaggtgcagtggctcacgcctatgatcccagcactttgggaggctgaggcgggtggatcacctgaggttaggagttcgagaccagcctggccaacatggtaaaaccccatctctactaaaaatacagaaattagccgggcgtggtggcgggcacctatagtcccagctactcagaagcctgaggctgggaaatcgtttgaacccgggaagcggaggttgcagggagccgagatcacgccactgcactccagcctgggcgacagagcgagagtctgtctcaaaagaaaaaaaaaagcaccgcctccaaatgccaacttgtccttttgtaccatggtgtgaaagtcagatgcccagagggcccaggcaggccaccatattcagtgctgtggcctgggcaagataacgcacttctaactagaaatctgccaattttttaaaaaagtaagtaccactcaggccaacaagccaacgacaaagccaaactctgccagccacatccaaccccccacctgccatttgcaccctccgccttcactccggtgtgcctgcagccccgcgcctccttccttgctgtcctaggccacaccatctcctttcagggaatttcaggaactagagatgactgagtcctcgtagccatctctctactcctacctcagcctagaccctcctcctcccccagaggggtgggttcctcttccccactccccaccttcaattcctgggccccaaacgggctgccctgccactttggtacatggccagtgtgatcccaagtgccagtcttgtgtctgcgtctgtgttgcgtgtcgtgggtgtgtgtagccaaggtcggtaagttgaatggcctgccttgaagccactgaagctgggattcctccccattagagtcagccttccccctcccagggccagggccctgcagaggggaaaccagtgtagccttgcccggattctgggaggaagcaggttgaggggctcctggaaaggctcagtctcaggagcatggggataaaggagaaggcatgaaattgtctagcagagcaggggcagggtgataaattgttgataaattccactggacttgagcttggcagctgaactattggagggtgggagagcccagccattaccatggagacaagaagggttttccaccctggaatcaagatgtcagactggctggctgcagtgacgtgcacctgtactcaggaggctgaggggaggatcactggagcccaggagtttgaggctgcagcgagctatgatcgcgccactacactccagcctgagcaacagagtgagaccctgtctcttaaagaaaaaaaaagtcagactgctgggactggccaggtttctgcccacattggacccacatgaggacatgatggagcgcacctgccccctggtggacagtcctgggagaacctcaggcttccttggcatcacagggcagagccgggaagcgatgaatttggagactctgtggggccttggttcccttgtgtgtgtgtgttgatcccaagacaatgaaagtttgcactgtatgctggacggcattcctgcttatcaataaacctgtttgttttaaaaaaaa

SEQ ID NO: 4 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 5 - DNA, 4468 bp, Artificial sequence
  Upstream homologous arm in targeting vector
agctcagttaccaccacccaagggatatacctaccccagggctggcagggcaagtgttcccatgagatgcacacaccgacagcccgtgtgtggagccggaggctgctgtttgaggaagtagaggagggtgaagagaaggttcttagttaatgagacaaatagtttccccaggaaggctagtctgggagttttattggggacactcaggccttctgtgtcagttagcattctttggagtgatggatacaaatgagctatgacactgatttctggggtccaaggctcccctaattctgctgtgtaccttcctcactcctttctcccaacttaccttactttctccatgggataggatactcagaccacattgagtagagcagaggagtctctaatccccaggaagagttctttcttattttatctttcctctttatgagcagtgtggctccccagcccagagactctccccccttagatgtgaaagggtcagccagccattgatgtaccctttgatgtgtttgttacattagcagttctcatctgaacacaacacactgggtccgagcggttccacgtgtgagatgtttattgggaggggggcgcaggggagaaggtgacagcagaaagagaaggggaaaaagagagagagggcctccttagaagtgggaacaaaacacccttggaaggagttacagagacaaagtttggagctgagattaaagggtggaccatgtagagactgccttatccagggatccaccccataatcagcatccaaacgctgacaccattgcatacactagcaagattttatcgaaaggacccagatgtagctgtctcttgtgagactatgccggggcctagcaaacacagaagtggatgcccacagtcagctaatggatggatcacagggctcccaatggaggagctagagaaagtacccaaggagctaaagggatctgcaaccctataggtggatcaacattatgaactaaccagtaccccggagctcttgactctagctgcacatgtatcaaaagatggcctagtcggccatcactggaaagagaggcccattggacacacaaactttatatgccccagaacaggggaatgccagggccaaaaagggggagtgggcaggtaggggagtgggggtgggtgggtatgggggacttttggtatagcattggaaatgtaaatgaactaaatacctaataaaaaatggaaaaaaaaaaaggaaaaaaaaagagatattaaggaaaagtaaaaaaaaaaaaagagagagagagagggagggctgttccccttatttatatgaaaaaaatgacgtaacacaggtaaaggtgggaggtgagccaagtggattctgggaatatgctgcaggtcgcgctgtcacctatgtgatgtcataggtttgggaggtcctgatgcaaagacccttgaccttgagaacagtcagtgaggtcccagacagtgtggaacacccttatttgtgtgctcagactcaactggttgttttgtcaagaaatggaacggggaagtgccaggatattgtgtgtaggcatgtgcgcatgcgcttggatgtgtgtgcagctgtccagagggagctgcagtggctttttcagactgggtactcagcatggttctgcgacttccctcacccagctgtctgcatgaccttcctgaaaaggattgccttgatggaggaactcagagataggccagggcagcagatgcatctatgttcaagcttcttggccctggtgcccagggggaggctctcgcctcaaggaaatgacattagaattattaccttactcagggtagcaactattgattactcttttctgtctggctctggatagggtagctcgcactgcagcctccaatgggccctcatctggggttactgggtgagcatgaagacaaggcagggggctggctgtattgggttgcttatccagggcacggtgtgcacagtcgcctgaagagggaagcactctgtgtggacttttggccaaaggcgcaagaggcagctagggtactgtggcaggtccagaagagccaaagactgtccacagcatagaaccccagtcccttgagtcatggcttctggtggctttcagctgttatcactgagtggctgagcaaagagaaacatgataggctcagaggtcactcagtgcacagcactaggcaaggtagggtgatgctctccagacacagctggggatgggaaggggaaagacagaaagcagagccactgcccacagatcatgggagtttgtgcagtctatacagaccaaatgagcctaatagtaaggtcggcatcttctctgattggcttgtttgcagtggcttttcagagactgatccatgcctttgctgtgctaattgttggatattttatgtatctctcttgctcccatagaattgagtcaggaaccgatgtccagagagatagggacacttgattgactatgaccatacagcttctggatggaagaataagaaaagaagaccacatctctctaactctccacctttatctgaggagtcaggggattcatttgggttactggtttaataaacatttattaagctcctgctgaccatatgacggcagctgctggtacaatcaagagtgcggagcagccttcctctttaacctagaaccaccccttggtcatttccaggtgacatcagcagaggctgcaagggtgttgagtgtgtgttcacaccctctttgctcttgtagatccactcccatgtttgctcatgagttcacatttgtgcagaaatggctaattcatgcttgtccctaaagtttccaattgctgttccaccttctggaagaaaggctggggcttaggcaggtctctgaggtactgggatagacccagtggtgacagttgctgctgctgtacacttctgagaactgaggacctcttggagatctgagggagggctctgtgtttctcacagttggttctgtgccatgttgggtgtttttttgttttttgttttttctcctccccatttgtctccctttcataattatttatttaattaaaaaatgtgagtactcctgagtatatgtatgtgaaccacacacattcaggagtatgcagaacttagaagggagcgtcaggttacctggaactggagtgacagacagttgtgagctgccatgttggtgctgggaactgatcccaagttgtctccaagaatagtaagtgcccctaaccactgcgctatctctggagctcctcctccccatcctccccatcgctccctgctttcctggtctctccctggaagtgcttagcccagtcattgaaatcatggcctagagagacagacaggacccaggctcaaatctctgtcactgccttaggtgggtaacctgtgctcgttatgcctgtttcctttctatacaatggtcggagtccaacatggctggttgttagagaactgaatgcaataatacatccatctcaggtccaggctctggaagcacagcctgaggcatggcttcttgtaacaggagaaggctctcaggcaagagggtggggtggcaggatgtggcagcagggtcacatgttttccagccaaaacctatgagtgttggtcttgggttgaggccaaggtactgcctttgagttggagacttgtcggccagtgctgaggctattctcctggcagttggagtctgagcatggctaggagcccccactggtccaaaccttctaagcccataggtgtaaggcagcactgatgtgaggaggtgcacaggtttgttttcattctgtattaacttcctgaggcagctctgccatggcctgtgtcagaagcttgggtctagaggtggcgcagccattatctgtgtcataggtggcagcatatggacacatgtatgtacacctgtgtgcatatgcatggatgtgagtgcaaggacacacggtgccaattcagaacaatgacatgaaggtcagaaggggccatgtctatattcagagagaacccagaggaggaagagcctacattaggcccaggagagtagagggtacttctgggagaaggaagacctcagccacaaggacagagggacagatggacagacatcctaaaaagagaagctttgtgtggaggtccttgtcacagtgagtcaagccactgtcttaaaaaaactacatcttctcagagccttgctgggtctcacccagcaggcaggagggaagccctaaagtaacccacttcctggcccagcaaactgcagacacaagcgtgccacgctgaagaggaaggccaagaagggaaaaagcctgggctctactctctcaagccttcctcgtcctccaacgcaaaaacccatccatgaggcctggtgacaggtatgctggagcccagagtcatactgatggctgcctcccttgttcccttccaggttgtcttgacaccctac

SEQ ID NO: 6 - DNA, 3487 bp, Artificial sequence
  Downstream homologous arm in targeting vector
agatgcaagtgtagaagagttgagtctccaaagatgtgatattgccatagggaatccctgggtggcatgaatgtgaacataggttctggttgctagttatgatgaacccgtttgtctcacagactatggactgctggtgtctctggcttccctctggggttaggatggtaccaagcccaaggccctacactgtagccttctgactgcatgtggtttgcagatgtgatctttggtttgaccctgaatttgagggcctaaggctggacttgatctcagcatcagcaccagccaccctggaacctttgtttctgagtaccctgccgttttcctaggtctgattgttggagtgacatcactgggtctgctgatgttaggactggtgaactgcatcatcctggtgcagaggaaaagtaaggttctgctctcgtcctgtttcccgccccacgtccctaccctaacactttctcggagccttgggtggcaggcctcgttcagagctctgcactatcatagactcgggtggatctggagactgtagcctcttgctctccaggaacttagagcctggtgtagaaggtacatactctaacctggctaccagaaggtagtgtcaaggctattccaggggattgaagttcaggatttttatacaggaagcctggagtctctcggattctgtgagctggcctgggtctcagtgggtggagtgtgttgaggcagggctacaacacaggctgtactggaggatggctggggcaaaggggtagtggctgaaagtagcatgggctcatccgtgaacatgacaggcagctactccctgggctgggtccactctcctggctctaccctagccatgctgtgcccattaagacagctgttctgaagaagtcctgcctctgacttgttcccctctcttcattgtagagaagccctcctgcctacaaagagatgccaaggtggtgagtatccctctgcggtcctcctcccccttctctcctccagctctccctcttcctcctcctctttctcctcctcttccagttcctttactagggcatcataagcaacatcatataagcaggatctaatgtatatggtggtgcatacctgccatcccaatagttaggggcaggtagaggctggagggtcagagtgtaaatactccctcagagccagctgatgtttagttccattacagttgggaagaaaccggcaaactctcccctagctgggcatagccaggctctttccctaggctatgtccaactcctcactggtcctatgcataaggcaccttggctcctcattatatagatgaggaaatgtaggcttaacagatacagtcacccttgaaaggttgtgttgtgtcctggtggtcaaaccctctgcgcatgtgcatccggcagctacacccaaagctgtctcaggctggcctctcctagcagggactgctcgccaagactggtcaccaggatttggagaacctagatgcccccctttgtccagccctgtgccctgtcagctacctgccccagggccctactctggagcccagtggacggtgtcctcatgtgctagagtgaggaggctgtcaggcaggagcagcagtgactcagcttctactctgaggactggggaagatgaggttatctggaaaaacagcacgtgaattcatctacacctcccccagtcatgcgcatggttcatgtttagtcgccggcagttactggtggtggtggaggaaggacgtgccagcactgtctgagggcttattcttccagcttgactgtgacacaggctatgacacaggctgtggcacaggctgtgacagaggttattgcccccaacgttagacagagtctgggattatatggctcagacactcttaagtaattgtcctgtgtcctagctgtgtcctagacacagctaatgggtatggcctggaacacaaacttagcatacaattactacccagcatacaattactgtgccactcttaggcatgcccacatgagcctgcacatataaatacccttagctcagaggccgacagaagccagccatgcttctgccagtcccagttccatataacaggaccagacccagaaccataggaggtatcaggcaagctaagattttttggtgttcattcagtggggtcctagccagagtcacatgactctgatagtgtctgtacttgaacactgtagcttgtggctttgaagaagctagctgtgacctttggggaggaggggaagatcgggttctgtctggctctagccacaagcagaaagggggaggcccagacagaaaagctccttctctggggctggtgtggtgtgacatttatgagtgcctctgtagagttacagggtaaggccagggccaaggtcagaccgtgagttcaggatgtttttcctctcttccattttggggtacctagctgggaggaccccaggggagtcaggaggatgcagtgggcatgctcccacatcctagactgggtttgcttactgtggccatcagccagctctgtaactctgagctctggattcatcttctgtgggcttttgtggcctcatgaagacagcttgtataagatccttccagtccagagtcagaagctgtggactgtgcaggaaacaaggcctcaaggccatgtatcatgacatacctgggcatctaagaagacatagcaacagtgggcaggacctggagatggaagttttagtgactctctagagagcaccagccacaggaaggatagcccaactattgtttcagcttagactacacctggcattgtgcctccctggaaaagtgtggccacttttccagctgctgtcaacagacaatggccttggaatagatgtccttagcataagttgccatgaacagggaatttgtcttaagcctcaagagctgaaccccagacacccagaagaggcatggtgagtttttagctcgactgcactgtatccaaaccatggcctcaggcagaaagttccgtttccatatttacagagctaggatggcactaggctatattgagtgtctagaagagctgagaccaatgtgactcaggcacaggatttggcactaggtgctcatgtcacatacagagtaagaagccacagggaagccacacgacatcatctgggtgaactgtgggaacgggcagctgagaagaccagtgaggaaccctgtgagtggccctgtgagtggccctgaggaaaggaccccatgggccagggtgattcattgagcagaaggaatgaagagtaaaggtgttaagcttcaagcaccaaggctggcctctgggacagctactctgatctgccacccaccatcaggaagtctgtgctttgcaaaacattgatgacagctcaggtgcatcaaacttgtttatttacgaacactaaaacttgacctttagatttgaggcattttgtattttagacacggacttatgtagcctgtattgacc

SEQ ID NO: 7 - DNA, 4275 bp, Artificial sequence
  human TNFR2 gene fragment
gccccggagcccgggagcacatgccggctcagagaatactatgaccagacagctcagatgtgctgcagcaaatgctcgccgggtgagggcagccacgggggcactcggggcccatgccctggaggagcgtgtgtgtacaggggctggggcgcaagagcatagcccttgattcttactgaactcctagttctatgcactggcccatgctccctgctgcttctgggcctgtgaacatgctgttccctcttcctggcacacttcccaactccctttccctgattaactccaacttgccctttagacctcagctgaagatgtcccctcttccaggaaggctgcctgccccatactgcccagccggtggaggagggggaggaggagcctaaattagctcctgggggagtcagggagggctgcctggaggaggcagcactcagcttgaaagacaagtgctgcctcctttgagctctcactggcctctgatgttcccatggaagctctttccttgctgtgggattatagcccggtcctgcttcctgttgcctccactgtgtgctcctctagggcagagattgtggcttgttttctgctgaatccccagcacgtggcgcaatgccatgtaggtgctcagtgcatatttgactgactctgtgaatgcttacccatttcacagatggtagacctgaggcccagccacagttggtggagctaggtagcctgactcctggtgccctccatctattcatttgtgtctgtattcattcatgtcatgattataaggtatctgctgtgtgttctaagtgctgggaatacacagagagcagggcagacaaaacccttgccctcgtggagtctgcattttaacggtggatgcagacagatacattaggagatgtgtggtcctgtctatctgtaagtgctgtagaaagacaaggtcaggtaaggcgggggagacagaacgggacatactgttttacacagggtggtctgggaaggtctctctgaggaggtgacatttgggcagagccctgcaggaggtgagggagcaggtcatgcatgtatacagggaacagtgtgccaggaggaagcaacagcatgtgcgaaggccttgaagtcggagcctgtttggtgagttggaggaacagcagggccagcatgtgtggcccaggatgagcgaaggcgagtacggtaggggaggaggaggaaggagtgagcagggaccatggtaaggagtttagattttattctgtctgggagccaatggagggctttgagccgttaccttgagatgtaggtgggcatatgagtacctgtcaccgtccctagccattttaagacatcctagggaaagtgactctcttctttcttttattaactggaagggtctctttaagaagttactagagaggctgaggtgggcggaccaccggaggccaggagtttgagaccagcctggccaacatggtggaacccgtctctactaagaatacaaaaaaatagctgggcataatggcaggcacctgtaatcccagctacttgggaggctgaggcaggagaattgcttgagcctgagaggcagaggttgcagtgagccgagattgtgccactgcactctagcctgggccacagagtgagattctgtctcaaaaaaaaaaaaaaaaaaaaaagcagctactgagtcaacttttttttaaggtttccgttggtgttctctgtccctggaatgcctgctttccaggcccatgcccttgtccttgataggataccagggactacattcttactacctgttccccctatcattcatgcccctagctggagaatattagccatctatcaagaaggaagaaactggcaagctagttcttactaaagtgtgattgactgataatagggctccagtggtggaggttcaagcatctggaaagagcagagaggaatctatttttgagcagaaggggttcagaaatgacgtgatagctggtacaatactagatgggagatgattctgaggaattggaactccacagaatagccttcccagctgggctttagaactctggactttgtggggacagtggatgagcccagggtcctggcagaaggctcgcccagctgagacctctggcccttgtttcctcaggccaacatgcaaaagtcttctgtaccaagacctcggacaccgtgtgtgactcctgtgaggacagcacatacacccagctctggaactgggttcccgagtgcttgagctgtggctcccgctgtagctctggtgagtaggttcagagaaaaagggggcccttacacccctgcctccaacttcccccggcaactccagcctctttggcttccagctgtctggagttaccccaggctggttgttggaagtggcacaggtgcagctgtttacccctaccactggcattttcctcctctgtctcaccaaagcctcttcacagccccacggggcaggcggtgggagaactgtgcccacgtgagggttgaggaggtggtgcgtgggagagtggtgcgcatgctcgtgctgcgaggagcaggactgcggggaggagcgaaactgctgggaagagcgggactgcggggaagagcgggacttcggggaggagcggcactgcggggaggagcaggactgcagggaggagcgggactgtggggaggagcgggacttcggggaggagcgggactgcgggggacgagcgcgactgcgggggacgagcaggactgcggagagtagcgggactgcggagagtagcgggactgcggagagtagcaggactgccggtcctgcccctggactctggccggtgttgtgtgtgccccatgctgaggcggtccgccagcctcctggagatccgctgtctgagagtgctgggctgtctgggagggcagcgtgggtgtggcggaggcaggcgtgaccgtttgccgccctctcgctgctctagaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggtacggggtggggctcaggtccttggggacgcccatgggcctctcctttgtagacatccttgcagtgtcacgggcatcaacccattaattagtccagcagggagcactgttagtggtggccaggtgcctgctacccatccgtctgtccacctgtccaccgttcattcttcctgccagcactcccgattaggcacctcttatgtactggaaccaggagacccagagatggatcagaccaagccctaagctaggaaagttatgtgatgctgcaagatgaactcacataactctacagcccaatcccgtgcatgtgtgtacaggaatctgtgtgtgtgcatgtgtgtacaggcatctgtgtgtgtgtgtgtgtgtgtgtgtgtgtgtaaggggtggaggtgcagacagagctccttgggcccctcagacctctcctagggctctagtgccaaggcccagctgtcccgcagagtgtctgagtggttgacaagttcggattgttccctgaaggaactgaaacatcagacgtggtgtgcaagccctgtgccccggggacgttctccaacacgacttcatccacggatatttgcaggccccaccagatgtgagtagctgagtcctttggttctggaggagcagggaggggctgtccctgggtgactgtgggtccaggacacagagcagctcaccaaccaccattgtccagactgctttatctgagggtggctcccaggataaatggcatggtgggcaggaccttgccctggaaggcaggactgggtgggtgcatggggaggtgggggagggctaggagggggtggtcctcagagagggcacacatcgtcactctcctatcctgcctgctggggcccgtgaatgagcccagccaccccagccactctgtcccctgctgcctcctgaccaagcctcctcctcctccagctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgct

SEQ ID NO: 8 - DNA, 4910 bp, Artificial sequence
  The mRNA sequence of the engineered mouse TNFR2 after humanization
agtcaccagctagagcgcagctgaggcactagagctccaggcacaagggcgggagccaccgctgcccctatggcgcccgccgccctctgggtcgcgctggtcttcgaactgcagctgtgggccaccgggcacacagtgcccgcccaggttgtcttgacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccaattggtctgattgttggagtgacatcactgggtctgctgatgttaggactggtgaactgcatcatcctggtgcagaggaaaaagaagccctcctgcctacaaagagatgccaaggtgcctcatgtgcctgatgagaaatcccaggatgcagtaggccttgagcagcagcacctgttgaccacagcacccagttccagcagcagctccctagagagctcagccagcgctggggaccgaagggcgccccctgggggccatccccaagcaagagtcatggcggaggcccaagggtttcaggaggcccgtgccagctccaggatttcagattcttcccacggaagccacgggacccacgtcaacgtcacctgcatcgtgaacgtctgtagcagctctgaccacagttctcagtgctcttcccaagccagcgccacagtgggagacccagatgccaagccctcagcgtccccaaaggatgagcaggtccccttctctcaggaggagtgtccgtctcagtccccgtgtgagactacagagacactgcagagccatgagaagcccttgccccttggtgtgccggatatgggcatgaagcccagccaagctggctggtttgatcagattgcagtcaaagtggcctgacccctgacaggggtaacaccctgcaaagggacccccgagaccctgaacccatggaacttcatgacttttgctggatccatttcccttagtggcttccagagccccagttgcaggtcaagtgagggctgagacagctagagtggtcaaaaactgccatggtgttttatgggggcagtcccaggaagttgttgctcttccatgacccctctggatctcctgggctcttgcctgattcttgcttctgagaggccccagtattttttccttctaaggagctaacatcctcttccatgaatagcacagctcttcagcctgaatgctgacactgcagggcggttccagcaagtaggagcaagtggtggcctggtagggcacagaggcccttcaggttagtgctaaactcttaggaagtaccctctccaagcccaccgaaattcttttgatgcaagaatcagaggccccatcaggcagagttgctctgttataggatggtagggctgtaactcagtggtccagtgtgcttttagcatgccctgggtttgatcctcagcaacacatgcaaaacgtaagtagacagcagacagcagacagcacagccagccccctgtgtggtttgcagcctctgcctttgacttttactctggtgggcacacagagggctggagctcctcctcctgaccttctaatgagcccttccaaggccacgccttccttcagggaatctcagggactgtagagttcccaggcccctgcagccacctgtctcttcctacctcagcctggagcactccctctaactccccaacggcttggtactgtacttgctgtgaccccaagtgcatgtccgggttaggcactgtgagttggaacagctgatgacatcggttgaaaggcccacccggaaacagctgaagccagctcttttgccaaaggattcatgccggttttctaatcaacctgctcccctagcatgcctggaaggaaagggttcaggagactcctcaagaagcaagttcagtctcaggtgcttggatgccatgctcaccgattccactggatatgaacttggcagaggagcctagttgttgccatggagacttaaagagctcagcactctggaatcaagatactggacacttggggccgacttgttaaggctctgcagcatcagactgtagaggggaaggaacacgtctgccccctggtggcccgtcctgggatgacctcgggcctcctaggcaacaaaagaatgaattggaaaggactgttcctgggtgtggcctcagctcctgtgcttgtgtggatccctaaagggtgtgctaaggagcaattgcactgtgtgctggacagaattcctgcttataaatgctttttgttgttgttttgtacactgagccctggctgagccaccccaccccacctcccatcccacctttacagccactcttgcagagaacctggctgtctcccacttgtagcctgtggatgctgaggaaacacccagccaagtagactccaggcttgcccctatctcctgctctgagtctggcctcctcattgtgttgtgggaaggagacgggttctgtcatctcggaagcccacaccgtggatgtgaacaatggctgtactagcttagaccagcttagggctctgcaatcagaggagggggagcagggaacaatttgagtgctgacctataacacattcctaaaggatgggcagtccagaatctccctccttcagtgtgtgtgtgtgtgtgtgtgtgtgtgtgtgtgtgtgtgtgtccatgtttgcatgtatgtgtgtgccagtgtgtggaggccagaggttggctttgggtgtgtttgatcactctcagttactgaggcagggctctcatctgtacccagagcttgcacattttctagtctaacttgcttcagggatctctgtctgcctatggagtgctcaggttacaggcaggctgccatacctgcccgacatttacatgaatactagagatctgaattctggtcctcacacttgtatacctgcattttatccactaagacatctctccaagggctcccccttcctatttaataagttagttttgaactggcaagatggctcagtgggtaaggcagtttgcggacaaacctgatgacctgagttggatccctgaccataaggtagaagagacctgattcctgcaagttgtcctctgaccaccaccccatacatgcttctgcatatgtgcacacatcacattcttgcacacacactcacataccataaatgtaataaatttttttaaataaattgattttatcttttaatcattatttttgcttatctgtttgttcatacatggagttttgctgtatacccagtctggctgcatcctccaggcaccttgaacttggatattctcctgcctcagtttcgagagtgctgagttataggcatgtgcaaccgtgtcaggacccttggcgttacattatgttttcgaatcttatttttccatctaagactgacaaccaccttgccaaaggatgaccttgacttttgtatcctcttgcctctacttcccaagtattgggatgacaagtctgtccatcctatgatttgtgtggtgctggggcttgaacccaggggcctcgtgaatgctaggcaaacgcaatcagctgcccaaccccgaacttcacttttcacctgtgatttcttaacatggttcctgagcagacagcatgatcctgttgccttgaggagctctgctcgttgctttgacaatgctctatttactaaagaatacttatagagttcaagactccaaagcagcagttcccaacctgtgggtatgacctcttgggtatcccatatcagatatttacattaaacttataacagaagcaaaattacagttatggggtagcaacaaaataatttttttttgaggcagggtttatctgtgtagctcactttgtacagactaggctggccttgaacttaaagatctgcctgcctctgccttctgaatgctgggattaaatgtgtgtgccaacatgcccagtgcagtgaaataattttattgttgggtcaccaaaacatgagaaagagtattaaaaggtcatagcgttaggaaggttgagaaccactgccccaaaagatagtagaaataggtaatagcattttaaaaaaagattaatttctttttccgtgtgtgtgtgtgtgtgtgtgtgtgtgtgtgtgtgtgtgtgtgtgtgtgtgtgtgaagggtgctgaattccctaaatctgggattacagacaattgtgccaaatgggtattgggaaccaaagttgggttttctgaaagagcagcaagtgcccttaactgctgagccctctctctagctataataaacttttttttaaaaaaattaaaaaaatttagcactc

SEQ ID NO: 9 - protein, 473 aa, Artificial sequence
  encoded protein sequence of the engineered mouse TNFR2 after humanization
MAPAALWVALVFELQLWATGHTVPAQVVLTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPIGLIVGVTSLGLLMLGLVNCIILVQRKKKPSCLQRDAKVPHVPDEKSQDAVGLEQQHLLTTAPSSSSSSLESSASAGDRRAPPGGHPQARVMAEAQGFQEARASSRISDSSHGSHGTHVNVTCIVNVCSSSDHSSQCSSQASATVGDPDAKPSASPKDEQVPFSQEECPSQSPCETTETLQSHEKPLPLGVPDMGMKPSQAGWFDQIAVKVA

SEQ ID NO: 10 - DNA, 70 bp, Artificial sequence
  connection between the 5′ end of the Neo cassette and the mouse TNFR2 gene locus
tccctttgggcagaactggggcctggtctctgtcttttagaacggaattccgaagttcctattctctaga

SEQ ID NO: 11 - DNA, 70 bp, Artificial sequence
  connection between the 3′ end of the Neo cassette with the mouse TNFR2 gene locus
aggaacttcatcagtcaggtacataatggtggatcccatatgagatgcaagtgtagaagagttgagtctc

SEQ ID NO: 12 - DNA, 25 bp, Artificial sequence
  primer
ctcgactgtgccttctagttgccag

SEQ ID NO: 13 - DNA, 25 bp, Artificial sequence
  primer
cctaacctctcttggtgctgagaac

SEQ ID NO: 14 - DNA, 25 bp, Artificial sequence
  primer
gatcagtgagacagtccaacttggc

SEQ ID NO: 15 - DNA, 24 bp, Artificial sequence
  primer
gcatgggccagtgcatagaactag

SEQ ID NO: 16 - DNA, 25 bp, Artificial sequence
  probe
tgatggtgggatgagtctgaagaag

SEQ ID NO: 17 - DNA, 25 bp, Artificial sequence
  probe
gaatgcctcaccctctctgctatta

SEQ ID NO: 18 - DNA, 25 bp, Artificial sequence
  probe
acctcgagtcagacttctgtaggta

SEQ ID NO: 19 - DNA, 25 bp, Artificial sequence
  probe
ctagggatataagcagaacgtggct

SEQ ID NO: 20 - DNA, 23 bp, Artificial sequence
  probe
ggatcggccattgaacaagatgg

SEQ ID NO: 21 - DNA, 23 bp, Artificial sequence
  probe
cagaagaactcgtcaagaaggcg

SEQ ID NO: 22 - DNA, 25 bp, Artificial sequence
  primer
tatgctggagcccagagtcatactg

SEQ ID NO: 23 - DNA, 25 bp, Artificial sequence
  primer
ggacatcattgcagtattgaggagg

SEQ ID NO: 24 - DNA, 24 bp, Artificial sequence
  primer
gcatgggccagtgcatagaactag

SEQ ID NO: 25 - DNA, 25 bp, Artificial sequence
  primer
tacttttaatgggagctgaggctgt

SEQ ID NO: 26 - DNA, 25 bp, Artificial sequence
  primer
atagtctgtgagacaaacgggttca

SEQ ID NO: 27 - DNA, 25 bp, Artificial sequence
  primer
gacaagcgttagtaggcacatatac

SEQ ID NO: 28 - DNA, 24 bp, Artificial sequence
  primer
gctccaatttcccacaacattagt

SEQ ID NO: 29 - DNA, 78 bp, Artificial sequence
  connection between the 3′end of the human TNFR2 gene sequence and the mouse TNFR2 gene locus
ggccccagccccccagctgaagggagcactggcgacttcgctcttccaattggtaagtcctcagtctcaagagtgacc

SEQ ID NO: 30 - DNA, 23 bp, Artificial sequence
  primer
gcctacaaagagatgccaaggtg

SEQ ID NO: 31 - DNA, 22 bp, Artificial sequence
  primer
tctgaaatcctggagctggcac

SEQ ID NO: 32 - protein, 474 aa, Mus musculus
MAPAALWVALVVELQLWATGHTVPAKVVLTPYKPEPGNQCQISQEYYDKKAQMCCAKCPPGQYAKHFCNKTSDTVCADCAAGMFTQVWNHLHTCLSCSSSCSDDQVETHNCTKKQNRVCACNADSYCALKLHSGNCRQCMKLSKCGPGFGVARSRTSNGNVICSACAPGTFSDTTSSTDVCRPHRICSILAIPGNASTDAVCASESPTPSAVPRTIYVSQPEPTRSQPMDQEPGPSQTPHIPVSLGSTPIIEPSITGGISLPIGLIVGLTTLGLLMLGLANCFILVQRKKKPSCLQRETMVPHLPDDKSQDAIGLEQQHLLTTAPSSSSSSLESSASAGDRRAPPGGHPQARVTAEAQGSQEACAGSRSSDSSHGSHGTHVNVTCIVNVCSSSDHSSQCSSQASTTVGDPDANPSGSPKDEQVPFSQEECPSQSQWETTETLQNHDKPFPLGVPDVGMKPNQPGWYDQIAVKVP